(19) United States
(12) Patent Application Publication
(10) Pub. No.: US 2026/0137793 A1
(43) Pub. Date: May 21, 2026

(54) Combination Therapies for Treating Cancer

(71) Applicant: GlaxoSmithKline Intellectual Property Development Limited, Stevenage (GB)

(72) Inventors: Matthew BRUCE, Collegeville, PA (US); Roxanne CACIOPPO, Waltham, MA (US); Ira GUPTA, Collegeville, PA (US); Brandon KREMER, Collegeville, PA (US); Jeffrey LIN, Collegeville, PA (US); Prani PAKA, Collegeville, PA (US); Antonio PALUMBO, Zug (CH); Christopher A. SHELTON, Collegeville, PA (US)

(73) Assignee: GlaxoSmithKline Intellectual Property Development Limited, Stevenage (GB)

(21) Appl. No.: 18/697,515

(22) PCT Filed: Oct. 4, 2022
(86) PCT No.: PCT/IB2022/059456

§ 371 Date: Apr. 1, 2024

Related U.S. Application Data
(60) Provisional application No. 63/252,479, filed on Oct. 5, 2021.

(51) Int. Cl. A61K 47/68; A61K 31/496; A61K 31/517; A61K 31/5377; A61P 35/00

(52) U.S. Cl. CPC A61K 47/68031; A61K 31/496; A61K 31/517; A61K 31/5377; A61K 47/6849; A61P 35/00

(57) ABSTRACT

Disclosed herein are methods and materials for treating cancer. The present disclosure further provides methods and materials for using one or more antigen binding proteins (for example anti-B-cell maturation antigen (BCMA) antigen binding proteins) and one or more cereblon E3 ligase modulators (CELMODs) for treating a subject having cancer.

### CROSS-REFERENCE TO RELATED APPLICATION

The subject application claims priority to U.S. Patent Application No. 63/252,479, filed on Oct. 5, 2021, the entirety of which is incorporated herein by reference.

### SEQUENCE LISTING

The instant application contains a Sequence Listing which has been submitted electronically in ASCII format and is hereby incorporated by reference in its entirety. Said ASCII copy, created on Sep. 22, 2022, is named 054624-09-5022-WO_Sequence_Listing.xml and is approximately 32.5 kilobytes in size.

### FIELD OF THE DISCLOSURE

The present disclosure relates to methods and materials for treating cancer. For example, the present disclosure provides methods and materials for using one or more antibody-drug conjugates (ADCs) and one or more cereblon E3 ligase modulators (CELMODs) for treating a mammal (e.g., a human) having cancer. The present disclosure further provides methods and materials for using one or more antigen binding proteins (for example anti-B-cell maturation antigen (BCMA) antigen binding protein) and one or more CELMODs for treating a subject having cancer.

### BACKGROUND INFORMATION

Multiple myeloma (MM) is an incurable malignancy and accounts for 1% of all cancers and for 10% of all hematologic malignancies. A variety of drugs and combination treatments have been evaluated and found effective in treating multiple myeloma. However, most, if not all, of these patients inevitably relapse.

Currently, there remains a need in the immunotherapy field for alternative or improved compositions and methods for more efficiently treating autoimmune disease and cancer.

### BRIEF SUMMARY

The present disclosure provides methods and materials for treating cancer. For example, the present disclosure provides methods and materials for using one or more molecules where each molecule includes: (i) an anti-BCMA antigen binding protein or antibody-drug conjugate (ADC) having binding specificity for a BCMA polypeptide and one or more cereblon E3 ligase modulator (CELMOD) for treating a subject having cancer. In some cases, a mammal (e.g., a human such as a human having cancer) can be administered a combination treatment disclosed herein comprising (a) an anti-BCMA antigen binding protein or ADC having binding specificity for a BCMA polypeptide and (b) one or more CELMODs.

Disclosed herein are combinations comprising an anti-BCMA antigen binding protein and a CELMOD. In some cases, the anti-BCMA antigen binding protein can comprise an antibody. In some cases, the antibody is a monoclonal antibody. In some cases, the monoclonal antibody is an IgG1. In some embodiments, the antibody is modified to enhance effector function. In some cases, the antibody is afucosylated. In some embodiments, the antibody is fucosylated. In some embodiments, the antibody is sialylated. In some embodiments, the antibody is glucosylated. In some embodiments, the antibody is glycosylated. In some embodiments, the antibody is galactosylated. In some cases, the anti-BCMA antigen binding protein is human, humanized or chimeric. In some cases, the anti-BCMA antigen binding protein can comprise a CDRH1 comprising the amino acid sequence set out in SEQ ID NO:1; a CDRH2 comprising the amino acid sequence set out in SEQ ID NO:2; a CDRH3 comprising the amino acid sequence set out in SEQ ID NO:3; a CDRL1 comprising the amino acid sequence set out in SEQ ID NO:4; a CDRL2 comprising the amino acid sequence set out in SEQ ID NO:5; and a CDRL3 comprising the amino acid sequence set out in SEQ ID NO:6. In some cases, the anti-BCMA antigen binding protein can comprise a heavy chain variable region (VH) comprising the amino acid sequence set out in SEQ ID NO:7; and a light chain variable region (VL) comprising the amino acid sequence set out in SEQ ID NO:8. In some cases, the anti-BCMA antigen binding protein can comprise a heavy chain (H) comprising the amino acid sequence set out in SEQ ID NO:9 and a light chain (L) comprising the amino acid sequence set out in SEQ ID NO: 10. In some cases, the anti-BCMA antigen binding protein is an immunoconjugate. In some cases, the anti-BCMA antigen binding protein is an immunoconjugate comprising an antibody conjugated to a cytotoxin. In some cases, the cytotoxin is MMAE or MMAF. In some cases, the cytotoxin is MMAF. In some embodiments, the cytotoxin is AFP, AEB, AEVB or auristatin E. In some embodiments, the cytotoxin is paclitaxel, docetaxel, CC-1065, SN-38, topotecan, morpholino-doxorubicin, rhizoxin, cyanomorpholino-doxorubicin, dolastatin-10, echinomycin, combretatstatin, calicheamicin, or netropsin. In some embodiments, the cytotoxin is an auristatin, a maytansinoid, or calicheamicin. In some embodiments, the cytotoxin is AFP, MMAP, vincristine, vinblastine, vindesine, vinorelbine, VP-16, camptothecin, paclitaxel, docetaxel, epothilone A, epothilone B, nocodazole, colchicines, colcimid, estramustine, cemadotin, discodermolide, maytansinol, maytansine, DM1, DM2, DM3, DM4 or eleutherobin. In some cases, the anti-BCMA antigen binding protein is belantamab mafodotin. In some cases, belantamab mafodotin is present in the combination at a dose of at least about 0.5 mg/kg, 0.95 mg/kg, 1.25 mg/kg, 1.4 mg/kg, 1.7 mg/kg, 2.5 mg/kg, or 3.4 mg/kg. In some embodiments, the therapeutically effective dose of the anti-BCMA antigen binding protein is 0.95 mg/kg. In some embodiments, the therapeutically effective dose of the anti-BCMA antigen binding protein is 1.0 mg/kg. In some embodiments, the therapeutically effective dose of the anti-BCMA antigen binding protein is 1.4 mg/kg. In some embodiments, the therapeutically effective dose of the anti-BCMA antigen binding protein is 1.9 mg/kg. In some embodiments, the therapeutically effective dose of the anti-BCMA antigen binding protein is 1.92 mg/kg. In some embodiments, the therapeutically effective dose of the anti-BCMA antigen binding protein is 2.5 mg/kg. In some embodiments, the therapeutically effective dose of the anti-BCMA antigen binding protein is 3.4 mg/kg. In some cases, the combination can comprise a pharmaceutically acceptable carrier. In some cases, the combination further comprising an adjuvant. In some embodiments, a CELMOD disclosed herein is mezigdomide (CC-92480), iberdomide (CC-220), avadomide (CC-122), CC-90009, CC-99282, a pharmaceutically acceptable salt of any of the foregoing, or any combination thereof. In some cases, a CELMOD disclosed herein is administered to the subject in a dose of at least about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, or 1.3 mg.

Disclosed herein are methods of treating cancer. In some cases, the method can comprise treating cancer in a subject in need thereof comprising administering to the subject a therapeutically effective dose of a combination disclosed herein. In some cases, the cancer is selected from the group consisting of multiple myeloma, chronic lymphocytic leukemia, Waldenstrom macroglobulinemia, and non-Hodgkin's lymphoma. In some cases, the cancer is multiple myeloma. In some cases, the cancer is relapsed and/or refractory multiple myeloma. In some cases, the subject has received at least one previous cancer treatment. In some cases, the therapeutically effective dose of the combination is administered to the subject at least about once every 1-60 days. In some cases, the therapeutically effective dose of the combination is administered to the subject at least about once every 3, 4, 6, or 8 weeks (e.g., 21 days). In some cases, the therapeutically effective dose of the combination is administered to the subject at least about once every 8 days. In some embodiments, the therapeutically effective dose of the anti-BCMA antigen binding protein is administered to the subject every week. In some embodiments, the therapeutically effective dose of the anti-BCMA antigen binding protein is administered to the subject every 2 weeks. In some embodiments, the therapeutically effective dose of the anti-BCMA antigen binding protein is administered to the subject every 3 weeks. In some embodiments, the therapeutically effective dose of the anti-BCMA antigen binding protein is administered to the subject every 4 weeks. In some embodiments, the therapeutically effective dose of the anti-BCMA antigen binding protein is administered to the subject every 5 weeks. In some embodiments, the therapeutically effective dose of the anti-BCMA antigen binding protein is administered to the subject every 6 weeks. In some embodiments, dosage of the therapeutically effective dose of the anti-BCMA antigen binding protein is step—down to a lower dose described herein following a first administration. In some cases, administering the therapeutically effective dose of the combination reduces ocular toxicity as compared to administering a therapeutically effective amount of the anti-BCMA antigen binding protein alone. In some cases, the anti-BCMA antigen binding protein is belantamab mafodotin. In some cases, ocular toxicity is at least one of: changes in corneal epithelium, dry eyes, irritation, redness, blurred vision, dry eyes, photophobia, or changes in visual acuity. In some cases, ocular toxicity is measured by at least one of the following methods: best corrected visual acuity, documentation of manifest refraction and the method used to obtain best corrected visual acuity, current glasses prescription (if applicable), intraocular pressure measurement, anterior segment (slit lamp) examination including fluorescein staining of the cornea and lens examination, dilated funduscopic examination, or an ocular surface disease index (OSDI). In some cases, an anti-BCMA antigen binding protein disclosed herein is administered to a subject in a dose of at least about 0.5 mg/kg, 0.95 mg/kg, 1.25 mg/kg, 1.4 mg/kg, 1.7 mg/kg, 2.5 mg/kg, 3.4 mg/kg or 4.6 mg/kg. In some embodiments, the therapeutically effective dose of the anti-BCMA antigen binding protein is 0.95 mg/kg. In some embodiments, the therapeutically effective dose of the anti-BCMA antigen binding protein is 1.0 mg/kg. In some embodiments, the therapeutically effective dose of the anti-BCMA antigen binding protein is 1.4 mg/kg. In some embodiments, the therapeutically effective dose of the anti-BCMA antigen binding protein is 1.9 mg/kg. In some embodiments, the therapeutically effective dose of the anti-BCMA antigen binding protein is 1.92 mg/kg. In some embodiments, the therapeutically effective dose of the anti-BCMA antigen binding protein is 2.5 mg/kg. In some embodiments, the therapeutically effective dose of the anti-BCMA antigen binding protein is 3.4 mg/kg. In some embodiments, the therapeutically effective dose of the anti-BCMA antigen binding protein is administered to the subject every week. In some embodiments, the therapeutically effective dose of the anti-BCMA antigen binding protein is administered to the subject every 2 weeks. In some embodiments, the therapeutically effective dose of the anti-BCMA antigen binding protein is administered to the subject every 3 weeks. In some embodiments, the therapeutically effective dose of the anti-BCMA antigen binding protein is administered to the subject every 4 weeks. In some embodiments, the therapeutically effective dose of the anti-BCMA antigen binding protein is administered to the subject every 5 weeks. In some embodiments, the therapeutically effective dose of the anti-BCMA antigen binding protein is administered to the subject every 6 weeks. In some embodiments, dosage of the therapeutically effective dose of the anti-BCMA antigen binding protein is step-down to a lower dose described herein following a first administration. In some embodiments, a 2.5 mg/kg or 3.4 mg/kg dosage of the therapeutically effective dose of the anti-BCMA antigen binding protein is step-down to a 1.9 mg/kg dose. In some embodiments, the therapeutically effective dose of the anti-BCMA antigen binding protein is administered to the subject on day 1, day 8 and thereafter every 3-12 weeks. In some cases, the combination further comprising an adjuvant. In some embodiments, a CELMOD disclosed herein is mezigdomide (CC-92480), iberdomide (CC-220), avadomide (CC-122), CC-90009, CC-99282, a pharmaceutically acceptable salt of any of the foregoing, or any combination thereof. In some cases, a CELMOD disclosed herein is administered to the subject in a dose of at least about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, or 1.3 mg.

Disclosed herein are the manufacture of a medicament for use. In some cases, disclosed herein are combinations for use in the manufacture of a medicament for treatment of cancer. Disclosed herein are the use of a combination disclosed herein for the treatment of cancer.

Disclosed herein are kits. In some cases, a kit disclosed herein is for use in treatment of cancer. In some cases, a kit disclosed herein can comprise a combination disclosed herein and instructions for use in the treatment of cancer.

Disclosed herein are pre-filled syringes or autoinjector devices. In some cases, a pre-filled syringe or autoinjector device disclosed herein can comprise a combination disclosed herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In addition, the materials, methods, and examples are illustrative only and not intended to be limiting.

The details of one or more embodiments of the invention are set forth in the accompanying description below. Other features, objects, and advantages of the invention will be apparent from the description and from the claims.

### DETAILED DESCRIPTION

### Combination

The present disclosure provides methods and materials for treating cancer. In some cases, disclosed herein is a combination comprising an anti-BCMA antigen binding protein and a CELMOD for use in treating cancer or other B-cell mediated diseases or disorders.

The term “combination” described herein refers to at least two therapeutic agents. As used herein the term “therapeutic agent” is understood to mean a substance that produces a desired effect in a tissue, system, animal, mammal, human, or other subject. In one embodiment, the combination can contain an additional therapeutic agent, such as, for example, an additional cancer therapeutic agent. In one embodiment, the additional cancer therapeutic may be an immunomodulatory imide drug (IMiD) such as thalidomide, lenalidomide, pomalidomide, apremilast, thalidomide or other thalidomide analog, bortezomib, dexamethasone, or a pharmaceutically acceptable salt thereof. In some embodiments, an additional cancer therapeutic may be carfilzomib, daratumumab, isatuximab, ixazomib, oprozomib, marizomib, or a pharmaceutically acceptable salt thereof. In some embodiments, an additional cancer therapeutic agent is a PD-1 inhibitor. In some cases, the PD-1 inhibitor is selected from the group consisting of PDR001, Nivolumab, Pembrolizumab, Pidilizumab, MED10680, REGN2810, TSR-042, PF-06801591, and AMP-224. In some cases, the PD-1 inhibitor is Jemperli. In some embodiments, an additional cancer therapeutic agent is a PD-L1 inhibitor. In some cases, the PD-L1 inhibitor is selected from the group consisting of FAZ053, Atezolizumab, Avelumab, Durvalumab, and BMS-93655. In some embodiments, an additional cancer therapeutic agent is a CTLA-4 inhibitor. In some cases, the CTLA-4 inhibitor is Ipilimumab or Tremelimumab. In some cases, an additional cancer therapeutic agent is a TIM⁻³ inhibitor. In some cases, the TIM⁻³ inhibitor is MGB453 or TSR-022. In some embodiments, an additional cancer therapeutic agent is a LAG-3 inhibitor. In some cases the LAG-3 inhibitor is selected from the group consisting of LAG525, BMS-986016, and TSR-033. In some embodiments, an additional cancer therapeutic agent is an mTOR inhibitor. In some cases the mTOR inhibitor is RAD001 or rapamycin.

The administration of the combinations disclosed herein may be advantageous over the individual therapeutic agents in that the combinations may provide one or more of the following improved properties when compared to the individual administration of a single therapeutic agent alone: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the therapeutic agents.

The combinations described herein can be in the form of a pharmaceutical composition. A “pharmaceutical composition” contains a combination described herein, and one or more pharmaceutically acceptable carriers, diluents, or excipients. The carrier(s), diluent(s) or excipient(s) must be acceptable in the sense of being compatible with the other ingredients of the formulation, capable of pharmaceutical formulation, and not deleterious to the recipient thereof. In one embodiment, each therapeutic agent in a combination can be individually formulated into its own pharmaceutical composition and each of the pharmaceutical compositions can be administered to treat cancer. In this embodiment, each of the pharmaceutical compositions may have the same or different carriers, diluents or excipients.

### Anti-BCMA Antigen Binding Protein

The term “anti-BCMA antigen binding protein” as used herein refers to antibodies and other protein constructs, such as domains, which are capable of binding to BCMA. The terms “BCMA binding protein” and “anti-BCMA antigen binding protein” are used interchangeably herein.

The anti-BCMA antigen binding proteins described herein may bind to human BCMA having, including, for example, human BCMA containing the amino acid sequence of GenBank Accession Number Q02223.2, or genes encoding human BCMA having at least 90 percent homology or at least 90 percent identity thereto.

Exemplary anti-BCMA antigen binding proteins and methods of making the same are disclosed in International Publication No. WO2012/163805 which is incorporated by reference herein in its entirety. Additional exemplary anti-BCMA antigen binding proteins include those described in WO2016/014789, WO2016/090320, WO2016/090327, WO2016/020332, WO2016/079177, WO2014/122143, WO2014/122144, WO2017/021450, WO2016/014565, WO2014/068079, WO2015/166649, WO2015/158671, WO2015/052536, WO2014/140248, WO2013/072415, WO2013/072406, WO2014/089335, US2017/165373, WO2013/154760, WO2018/201051 and WO2017/051068, each of which is incorporated by reference herein in its entirety.

The term “antigen binding protein” as used herein refers to antibodies and other protein constructs, such as domains, which are capable of binding to the antigen.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., a domain antibody (DAB)), antigen binding antibody fragments, Fab, F(ab′)₂, Fv, disulphide linked Fv, single chain Fv, disulphide-linked scFv, diabodies, TANDABS, etc. and modified versions of any of the foregoing.

In some embodiments, a BCMA binding protein disclosed herein may be derived from rat, mouse, primate (e.g., cynomolgus, Old World monkey or Great Ape) or human. The BCMA binding protein may be a human, humanized or chimeric antibody. The BCMA binding protein may comprise a constant region, which may be of any isotype or subclass. The constant region may be of the IgG isotype, for example IgG1, IgG2, IgG3, IgG4 or variants thereof. The BCMA binding protein constant region may be IgG1.

The term, “full”, “whole” or “intact” antibody, used interchangeably herein, refers to a heterotetrameric glycoprotein. An intact antibody can be composed of two identical heavy chains (HCs) and two identical light chains (LCs) linked by covalent disulphide bonds. This H₂L₂ structure folds to form three functional domains comprising two antigen-binding fragments, known as ‘Fab’ fragments, and a ‘Fc’ crystallisable fragment. The Fab fragment can be composed of the variable domain at the amino-terminus, variable heavy (VH) or variable light (VL), and the constant domain at the carboxyl terminus, CH1 (heavy) and CL (light). The Fc fragment can be composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a κ or λ light chain. The majority of antibodies in the serum belong to the IgG class, and there are four isotypes of human IgG (IgG1, IgG2, IgG3 and IgG4), the sequences of which differ mainly in their hinge region.

As used herein, “about” means plus or minus 10%.

Fully human antibodies can be obtained using a variety of methods, for example using yeast-based libraries or transgenic animals (e.g., mice) that can produce repertoires of human antibodies. Yeast presenting human antibodies on their surface that bind to an antigen of interest can be selected using FACS (Fluorescence-Activated Cell Sorting) based methods or by capture on beads using labelled antigens. Transgenic animals that have been modified to express human immunoglobulin genes can be immunized with an antigen of interest and antigen-specific human antibodies isolated using B-cell sorting techniques. Human antibodies produced using these techniques can then be characterized for desired properties such as affinity, developability and selectivity.

In some aspects, alternative antibody formats can be used. Alternative antibody formats include alternative scaffolds in which the one or more CDRs of the BCMA antibody can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain.

The term “domain” refers to a folded polypeptide structure which retains its tertiary structure independent of the rest of the polypeptide. Generally, domains are responsible for discrete functional properties of polypeptides and in many cases may be added, removed or transferred to other polypeptides without loss of function of the remainder of the protein and/or of the domain.

The term “single variable domain” refers to a folded polypeptide domain comprising sequences characteristic of antibody variable domains. It can include complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain can bind an antigen or epitope independently of a different variable region or domain. A “domain antibody” or “DAB” may be considered the same as a “single variable domain”. A single variable domain may be a human single variable domain, but may also include single variable domains from other species such as rodent (for example, as disclosed in WO 00/29004 A1), nurse shark and Camelid VHH DABs. Camelid VHH are immunoglobulin single variable domain polypeptides that are derived from species including camel, llama, alpaca, dromedary, and guanaco, which produce heavy chain antibodies naturally devoid of light chains. Such VHH domains may be humanized according to standard techniques available in the art, and such domains can be considered “single variable domains”. As used herein, VH includes camelid VHH domains.

An antigen binding fragment, BCMA binding protein fragment, functional fragment, biologically active fragment or an immunologically effective fragment may comprise partial heavy or light chain variable sequences. Fragments can be at least: 5, 6, 8 or 10 amino acids in length. Alternatively, the fragments can be at least 15, at least 20, at least 50, at least 75, or at least 100 amino acids in length.

An antigen binding fragment may be provided by means of arrangement of one or more CDRs on non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example an IgG, or IgA scaffold. The IgG scaffold may comprise some or all the domains of an antibody (i.e. CH1, CH2, CH3, VH, VL). An antigen binding protein disclosed herein may comprise an IgG scaffold selected from IgG1, IgG2, IgG3, IgG4 or IgG4PE. For example, the scaffold may be IgG1. The scaffold may consist of, or comprise, the Fc region of an antibody, or be a part thereof.

The protein scaffold may be a derivative of a scaffold selected from the group consisting of CTLA-4, lipocalin, Protein A derived molecules such as Z-domain of Protein A (Affibody, SpA), A-domain (Avimer/Maxibody); heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxin kunitz type domains of human protease inhibitors; and fibronectin/adnectin; which has been subjected to protein engineering in order to obtain binding to an antigen other than the natural ligand.

The term “antigen binding site” refers to a site on an antigen binding protein which is capable of specifically binding to an antigen, this may be a single variable domain, or it may be paired VH/VL domains as can be found on a standard antibody. Single-chain Fv (ScFv) domains can also provide antigen-binding sites.

The term multi-specific antigen binding protein refers to an antigen binding protein that comprises at least two different antigen binding sites. Each of these antigen-binding sites is capable of binding to a different epitope, which may be present on the same antigen or different antigens. The multi-specific antigen binding protein may have specificity for more than one antigen, for example two antigens, or three antigens, or four antigens.

Classification and formats of bispecific antibodies are comprehensively described in reviews by Labrijn et al 2019 and Brinkmann and Kontermann 2017. Bispecifics may be generally classified as having a symmetric or asymmetric architecture. Bispecifics may have an Fc or may be fragment-based (lacking an Fc). Fragment based bispecifics combine multiple antigen-binding antibody fragments in one molecule without an Fc region e.g. Fab-scFv, Fab-scFv₂, orthoganol Fab-Fab, Fab-Fv, tandem scFc (e.g. BiTE and BiKE molecules), Diabody, DART, TandAb, scDiabody, tandem dAb etc.

Symmetric formats combine multiple binding specificities in a single polypeptide chain or single HL pair including Fc-fusion proteins of fragment-based formats and formats whereby antibody fragments are fused to regular antibody molecules. Examples of symmetric formats may include DVD-Ig, TVD-Ig, CODV-Ig, (scFv)4-Fc, IgG-(scFv)2, Tetravalent DART-Fc, F(ab)₄CrossMab, IgG-HC-scFv, IgG-LC-scFv, mAb-dAb etc.

Asymmetric formats retain as closely as possible the native architecture of natural antibodies by forcing correct HL chain pairing and/or promoting H chain heterodimerization during the co-expression of three (if common heavy or light chains are used) or four polypeptide chains e.g. Triomab, asymmetric reengineering technology immunoglobulin (ART-Ig), CrossMab, Biclonics common light chain, ZW1 common light chain, DuoBody and knobs into holes (KiH), DuetMab, KA body, Xmab, YBODY, HET-mAb, HET-Fab, DART-Fc, SEEDbody, mouse/rat chimeric IgG.

Bispecific formats also include an antibody fused to a non-Ig scaffold such as Affimabs, Fynomabs, Zybodies, and Anticalin-IgG fusions, ImmTAC. In some embodiments, an antigen binding protein described herein is a multi-specific antigen binding protein.

The term “chimeric antigen receptor” (“CAR”) as used herein, refers to an engineered receptor which consists of an extracellular antigen binding domain (which is usually derived from a monoclonal antibody, or fragment thereof, e.g., a VH domain and a VL domain in the form of a scFv), optionally a spacer region, a transmembrane region, and one or more intracellular effector domains. CARs have also been referred to as chimeric T cell receptors or chimeric immunoreceptors (CIRs). CARs can be genetically introduced into hematopoietic cells, such as T cells, to redirect T cell specificity for a desired cell-surface antigen, resulting in a CAR-T therapeutic. In some embodiments, a CAR comprises an anti-BCMA antigen binding protein disclosed herein.

The term “spacer region” as used herein, refers to an oligo- or polypeptide that functions to link the transmembrane domain to the target binding domain. This region may also be referred to as a “hinge region” or “stalk region”. The size of the spacer can be varied depending on the position of the target epitope in order to maintain a set distance (e.g., 14 nm) upon CAR:target binding.

The term “transmembrane domain” as used herein refers to the part of the CAR molecule which traverses the cell membrane.

The term “intracellular effector domain” (also referred to as the “signaling domain”) as used herein refers to the domain in the CAR which can be responsible for intracellular signaling following the binding of the antigen binding domain to the target. The intracellular effector domain can be responsible for the activation of at least one of the normal effector functions of the immune cell in which the CAR is expressed. For example, the effector function of a T cell can be a cytolytic activity or helper activity including the secretion of cytokines.

It will be appreciated by a person skilled in the art that VH and/or VL domains disclosed herein may be incorporated, e.g., in the form of a scFv, into CAR-T therapeutics.

Affinity, also referred to as “binding affinity”, is the strength of binding at a single interaction site, i.e., of one molecule, e.g., a BCMA binding protein disclosed herein, to another molecule, e.g., its target antigen, at a single binding site. The binding affinity of an antigen binding protein to its target may be determined by equilibrium methods (e.g., enzyme-linked immunoabsorbent assay (ELISA) or radioimmunoassay (RIA)), or kinetics (e.g., BIACORE analysis).

Avidity, also referred to as functional affinity, is the cumulative strength of binding at multiple interaction sites, e.g., the sum total of the strength of binding of two molecules (or more, e.g., in the case of a bispecific or multispecific molecule) to one another at multiple sites, e.g., taking into account the valency of the interaction.

In an embodiment, the equilibrium dissociation constant (KD) of an antigen binding protein disclosed herein—antigen interaction can be 100 nM or less, 10 nM or less, 2 nM or less or 1 nM or less. Alternatively, the KD may be between 5 and 10 nM; or between 1 and 2 nm. The KD may be between 1 pM and 500 pM; or between 500 pM and 1 nM. A skilled person will appreciate that the smaller the KD numerical value, the stronger the binding. The reciprocal of KD (i.e. 1/KD) is the equilibrium association constant (KA) having units M⁻¹. A skilled person will appreciate that the larger the KA numerical value, the stronger the binding.

The dissociation rate constant (kd) or “off-rate” describes the stability of the antigen binding protein—complex, i.e. the fraction of complexes that decay per second. For example, a kd of 0.01 s⁻¹ equates to 1% of the complexes decaying per second. In an embodiment, the dissociation rate constant (kd) can be 1×10⁻³ s⁻¹ or less, 1×10⁻⁴ s⁻¹ or less, 1×10⁻⁵ s⁻¹ or less, or 1×10⁻⁶ s⁻¹ or less. The kd may be between 1×10⁻⁵ s⁻¹ and 1×10⁻⁴ s⁻¹; or between 1×10⁻⁴ s⁻¹ and 1×10⁻³ s⁻¹. In some embodiments, the kd of an antigen binding protein disclosed herein can be 2.06×10⁻⁴ s⁻¹ or less, 1.58×10⁻⁴ s⁻¹ or less, 1.7×10⁻⁴ s⁻¹ or less, or 5.68×10⁻⁴ s⁻¹ or less, 6.78×10⁻⁴ s⁻¹ or less, 8.26×10⁻⁴ s⁻¹ or less, 5.15×10⁻⁴ s⁻¹ or less, or 5.68×10⁻⁴ s⁻¹ or less.

The association rate constant (ka) or “on-rate” describes the rate of antigen binding protein-complex formation. In an embodiment, the association rate constant (ka) can be 6.49×10⁶ M⁻¹s⁻¹, 4.65×10⁶ M⁻¹s⁻¹, 3.27×10⁶ M⁻¹s⁻¹, 8.28×10⁶ M⁻¹s⁻¹, 1.47×10⁷ M⁻¹s⁻¹, 1.10×10⁷ M⁻¹s⁻¹, or 5.90×10⁶ M⁻¹s⁻¹.

It will be apparent to those skilled in the art that the term “derived” is intended to define not only the source in the sense of it being the physical origin for the material but can also define material which is structurally identical to the material but which does not originate from the reference source.

By “isolated” it is intended that the molecule, such as a BCMA binding protein, is removed from the environment in which it may be found in nature. For example, the molecule may be purified away from substances with which it would normally exist in nature. For example, the BCMA binding protein can be purified to at least 95%, 96%, 97%, 98% or 99%, or greater with respect to a culture media containing the BCMA binding protein. The BCMA binding proteins and antibodies disclosed herein may be isolated BCMA binding proteins and antibodies.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g., within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987).

### Variants

It will be apparent to those skilled in the art that there are alternative numbering conventions for amino acid residues in variable domain sequences and full-length antibody sequences. There are also alternative numbering conventions for CDR sequences, for example those set out in Chothia et al. (1989) Nature 342: 877-883. For example, the structure and protein folding of a BCMA binding protein may mean that other residues can be considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1
Exemplary numbering convention for each CDR or binding unit.

| | | | | | Minimum |
|---|---|---|---|---|---|
| | Kabat CDR | Chothia CDR | AbM CDR | Contact CDR | Binding Unit |
| H1 | 31-35/35A/35B | 26-32/33/34 | 26-35/35A/35B | 30-35/35A/35B | 31-32 |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

Accordingly, a BCMA binding protein is provided, which can comprise any one or a combination of the following CDRs: CDRH1 of SEQ ID NO: 1, CDRH2 of SEQ ID NO: 2, CDRH3 of SEQ ID NO: 3, CDRL1 of SEQ ID NO: 4, CDRL2 of SEQ ID NO: 5, CDRL3 of SEQ ID NO: 6. CDRs may be modified by at least one amino acid substitution, deletion or addition, wherein the variant antigen binding protein substantially retains the biological characteristics of the unmodified protein, such as binding to the antigen.

TABLE 2
Exemplary CDR sequences for an anti-BCMA antigen
binding protein.

| | Sequence | SEQ ID NO |
|---|---|---|
| V_{H} CDR1 | NYWMH | 1 |
| V_{H} CDR2 | ATYRGHSDTYYNQKFKG | 2 |
| V_{H} CDR3 | GAIYDGYDVLDN | 3 |
| V_{L} CDR1 | SASQDISNYLN | 4 |
| V_{L} CDR2 | YTSNLHS | 5 |
| V_{L} CDR3 | QQYRKLPWT | 6 |

It will be appreciated that each of CDR H1, H2, H3, L1, L2, L3 may be modified alone or in combination with any other CDR, in any permutation or combination. In one embodiment, a CDR can be modified by the substitution, deletion or addition of up to 3 amino acids, for example 1 or 2 amino acids, for example 1 amino acid. Typically, the modification can be a substitution, particularly a conservative substitution, for example as shown in Table 3 below.

TABLE 3
Exemplary Substitutions.

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain | Gly, Pro |
| orientation | |
| Aromatic | Trp, Tyr, Phe |

For example, in a variant CDR, the flanking residues that comprise the CDR as part of alternative definition(s) e.g., Kabat or Chothia, may be substituted with a conservative amino acid residue.

The VH or VL (or HC or LC) sequence disclosed herein may be a variant sequence with up to 10 amino acid substitutions, additions or deletions. For example, the variant sequence may have up to 9, 8, 7, 6, 5, 4, 3, 2 or 1 amino acid substitution(s), addition(s) or deletion(s). The sequence variation may exclude one or more or all of the CDRs, for example the CDRs are the same as the VH or VL (or HC or LC) sequence and the variation is in the remaining portion of the VH or VL (or HC or LC) sequence, so that the CDR sequences are fixed and intact.

Alternatively, the heavy chain variable region may have 75% or greater, 80% or greater, 85% or greater, 90% or greater, 95% or greater, 98% or greater, 99% or greater or 100% identity to an amino acid sequence described herein for an antibody; and the light chain variable region may have 75% or greater, 80% or greater, 85% or greater, 90% or greater, 95% or greater, 98% or greater, 99% or greater, or 100% identity to an amino acid sequence disclosed herein for an antibody.

The heavy chain variable region of an antibody or amino acid sequence disclosed herein may be a variant which may contain 30, 25, 20, 15, 10, 9, 8, 7, 6, 5, 4, 3, 2 or 1 amino acid substitutions, insertions or deletions. The light chain variable region of an antibody or amino acid sequence disclosed herein may be a variant which may contain 30, 25, 20, 15, 10, 9, 8, 7, 6, 5, 4, 3, 2 or 1 amino acid substitutions, insertions or deletions.

The term “epitope” as used herein refers to that portion of the antigen contacting a particular binding domain of an antigen binding protein, also known as the paratope. An epitope may be linear or conformational/discontinuous. A conformational or discontinuous epitope can comprise amino acid residues that are separated by other sequences, i.e., not in a continuous sequence in the antigen's primary sequence assembled by tertiary folding of the polypeptide chain. Although the residues may be from different regions of the polypeptide chain, they are in close proximity in the three dimensional structure of the antigen. In the case of multimeric antigens, a conformational or discontinuous epitope may include residues from different peptide chains. Particular residues comprised within an epitope can be determined through computer modelling programs or via three-dimensional structures obtained through methods known in the art, such as X-ray crystallography. Exemplary methods include peptide based approaches such as pepscan whereby a series of overlapping peptides are screened for binding using techniques such as ELISA or by in vitro display of large libraries of peptides or protein mutants, e.g., on phage. Detailed epitope information can be determined by structural techniques including X-ray crystallography, solution nuclear magnetic resonance (NMR) spectroscopy and cryogenic-electron microscopy (cryo-EM). Mutagenesis, such as alanine scanning, can be an effective approach whereby loss of binding analysis is used for epitope mapping. Another method is hydrogen/deuterium exchange (HDX) combined with proteolysis and liquid-chromatography mass spectrometry (LC-MS) analysis to characterize discontinuous or conformational epitopes.

### Percent Identity

“Percent identity” between a query nucleic acid sequence and a subject nucleic acid sequence is the “Identities” value, expressed as a percentage, that is calculated using a suitable algorithm or software, such as BLASTN, FASTA, DNASTAR Lasergene, GeneDoc, Bioedit, EMBOSS needle or EMBOSS infoalign, over the entire length of the query sequence after a pair-wise global sequence alignment has been performed using a suitable algorithm or software, such as BLASTN, FASTA, ClustalW, MUSCLE, MAFFT, EMBOSS Needle, T-Coffee, and DNASTAR Lasergene. In some cases, a query nucleic acid sequence may be described by a nucleic acid sequence identified in one or more claims herein.

“Percent identity” between a query amino acid sequence and a subject amino acid sequence is the “Identities” value, expressed as a percentage, that is calculated using a suitable algorithm or software, such as BLASTP, FASTA, DNASTAR Lasergene, GeneDoc, Bioedit, EMBOSS needle or EMBOSS infoalign, over the entire length of the query sequence after a pair-wise global sequence alignment has been performed using a suitable algorithm/software such as BLASTP, FASTA, ClustalW, MUSCLE, MAFFT, EMBOSS Needle, T-Coffee, and DNASTAR Lasergene. In some cases, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity can be less than 100%. For example, the query sequence can be at least: 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDRs. Alternatively, the % identity may exclude one or more or all of the CDRs, for example all of the CDRs may be 100% identical to the subject sequence and the % identity variation may be in the remaining portion of the query sequence, e.g., the framework sequence, so that the CDR sequences may be fixed and intact. In some embodiments, an anti-BCMA binding protein disclosed herein comprises a sequence that is at least about 50, 60, 70, 75, 80, 85, 90, 95, 96, 97, 98, or 99% identical to a sequence disclosed herein.

### Modifications

The terms “peptide”, “polypeptide” and “protein” each refers to a molecule comprising two or more amino acid residues. A peptide may be monomeric or polymeric. A peptide disclosed herein can be modified.

Fc engineering methods can be applied to modify the functional or pharmacokinetics properties of an antibody. Effector function may be altered by making mutations in the Fc region that increase or decrease binding to C1q or Fcγ receptors and modify CDC or ADCC activity respectively. Modifications to the glycosylation pattern of an antibody can also be made to change the effector function. The in vivo half-life of an antibody can be altered by making mutations that affect binding of the Fc to the FcRn (Neonatal Fc Receptor).

### Effector Function

The term “Effector Function” as used herein refers to one or more of antibody-mediated effects including antibody-dependent cell-mediated cytotoxicity (ADCC), antibody-mediated complement activation including complement-dependent cytotoxicity (CDC), complement-dependent cell-mediated phagocytosis (CDCP), antibody dependent complement-mediated cell lysis (ADCML), and Fc-mediated phagocytosis or antibody-dependent cellular phagocytosis (ADCP).

The interaction between the Fc region of an antigen binding protein or antibody and various Fc receptors (FcR), including FcγRI (CD64), FcγRII (CD32), FcγRIII (CD16), FcRn, C1q, and type II Fc receptors is believed to mediate the effector functions of the antigen binding protein or antibody. Significant biological effects can be a consequence of effector functionality. Usually, the ability to mediate effector function requires binding of the antigen binding protein or antibody to an antigen and not all antigen binding proteins or antibodies will mediate every effector function.

Effector function can be assessed in a number of ways including, for example, evaluating ADCC effector function of antibody coated to target cells mediated by Natural Killer (NK) cells via FcγRIII, or monocytes/macrophages via FcγRI, or evaluating CDC effector function of antibody coated to target cells mediated by complement cascade via C1q. For example, an antigen binding protein of the present invention can be assessed for ADCC effector function in a Natural Killer cell assay. Examples of such assays can be found in Shields et al, 2001, The Journal of Biological Chemistry, Vol. 276, p. 6591-6604; Chappel et al, 1993, The Journal of Biological Chemistry, Vol 268, p. 25124-25131; Lazar et al, 2006, PNAS, 103; 4005-4010. Examples of assays to determine CDC function include those described in J Imm Meth, 1995, 184: 29-38. The effects of mutations on effector functions (e.g., FcRn binding, FcγRs and C1q binding, CDC, ADCML, ADCC, ADCP) can be assessed, e.g., as described in Grevys et al., J Immunol. 2015 Jun. 1; 194(11): 5497-5508, or Tam et al., Antibodies 2017, 6(3); Monnet et al., 2014 mAbs, 6:2, 422-436. Throughout this specification, amino acid residues in Fc regions, in antibody sequences or full-length antigen binding protein sequences, are numbered according to the EU index numbering convention.

### Enhancement

Human IgG1 constant regions containing specific mutations have been shown to enhance binding to Fc receptors. In some cases these mutations have also been shown to enhance effector functions, such as ADCC and CDC, as described below. Antigen binding proteins of the present invention may include any of the following mutations.

Enhanced CDC: Fc engineering can be used to enhance complement-based effector function. For example (with reference to IgG1), K326W/E333S; S267E/H268F/S324T; and IgG1/IgG3 cross subclass can increase C1q binding; E345R (Diebolder et al., Science 2014; 343: 1260-1293) and E345R/E430G/S440Y results in preformed IgG hexamers (Wang et al., Protein Cell. 2018 January; 9(1): 63-73).

Enhanced ADCC: Fc engineering can be used to enhance ADCC. For example (with reference to IgG1), F243L/R292P/Y300L/V3051/P396L; S239D/1332E; and S298A/E333A/K334A increase FcγRIIIa binding; S239D/1332E/A330L increases FcγRIIIa binding and decreases FcγRIIb binding; G236A/S239D/1332E improves binding to FcγRIIa, improves the FcγRIIa/FcγRIIb binding ratio (activating/inhibitory ratio), and enhances phagocytosis of antibody-coated target cells by macrophages. An asymmetric Fc in which one heavy chain contains L234Y/L235Q/G236W/S239M/H268D/D270E/S298A mutations and D270E/K326D/A330M/K334E in the opposing heavy chain, increases affinity for FcγRIIIa F158 (a lower-affinity allele) and FcγRIIIa V158 (a higher-affinity allele) with no increased binding affinity to inhibitory FcγRIIb (Mimoto et al., 2013).

Enhanced ADCP: Fc engineering can be used to enhance ADCP. For example (with reference to IgG1), G236A/S239D/1332E increases FcγRIIa binding and increases FcγRIIIa binding (Richards J et al., Mol. Cancer Ther. 2008; 7: 2517-2527).

Increased co-engagement: Fc engineering can be used to increase co-engagement with FcRs. For example (with reference to IgG1), S267E/L328F increases FcγRIIb binding; N325S/L328F increases FcγRIIa binding and decreases FcγRIIIa binding (Wang et al. 2018).

### Glycosylation

An antigen binding protein of the present invention may comprise a heavy chain constant region with an altered glycosylation profile, such that the antigen binding protein has an enhanced effector function, e.g. enhanced ADCC, enhanced CDC, or both enhanced ADCC and CDC. Examples of suitable methodologies to produce antigen binding proteins with an altered glycosylation profile are described in WO2003011878, WO2006014679 and EP1229125, all of which can be applied to the antigen binding proteins of the present invention.

The absence of the α1,6 innermost fucose residues on the Fc glycan moiety on N297 of IgG1 antibodies enhances affinity for FcγRIIIA. As such, afucosylated or low fucosylated monoclonal antibodies may have increased therapeutic efficacy (Shields et al., J Biol Chem. 2002, 277(30): 26733-40 and Monnet et al., 2014, mAbs, 6:2, 422-436).

### Potelligent

The present invention also provides a method of producing an antigen binding protein according to the invention comprising the steps of:
a) culturing a recombinant host cell comprising an expression vector comprising the isolated nucleic acid as described herein, wherein the FUT8 gene encoding alpha-1,6-fucosyltransferase has been inactivated in the recombinant host cell; and
b) recovering the antigen binding protein.

Such methods for the production of antigen binding proteins can be performed, for example, using the POTELLIGENT technology system available from BioWa, Inc. (Princeton, NJ) in which CHOK1SV cells lacking a functional copy of the FUT8 gene produce monoclonal antibodies having enhanced ADCC activity that is increased relative to an identical monoclonal antibody produced in a cell with a functional FUT8 gene as described in U.S. Pat. Nos. 7,214,775, 6,946,292, WO0061739 and WO0231240, all of which are incorporated herein by reference. Those of ordinary skill in the art will also recognize other appropriate systems.

In an embodiment of the invention, the antigen binding protein is produced in a host cell in which the FUT8 gene has been inactivated. In an embodiment of the invention, the antigen binding protein is produced in a −/−FUT8 host cell. In an embodiment of the invention, the antigen binding protein is afucosylated at Asn297 (IgG1).

### Other Modifications

In some embodiments, it may be desirable to modify the effector function of an antigen binding protein disclosed herein, for instance, to enhance ADCC or CDC, half life, etc. In an embodiment, an antigen binding protein may be Fc disabled. One way to achieve Fc disablement can comprise the substitutions of alanine residues at positions 235 and 237 (EU index numbering) of the heavy chain constant region. Alternatively, an antigen binding protein may be Fc enabled and not comprise the alanine substitutions at positions 235 and 237. An antigen binding protein may have a half life of at least 6 hours, at least 1 day, at least 2 days, at least 3 days, at least 4 days, at least 5 days, at least 7 days, or at least 9 days in vivo in humans, or in a murine animal model.

Mutational changes to the Fc effector portion of the antibody can be used to change the affinity of the interaction between the FcRn and antibody to modulate antibody turnover. The half-life of the antibody can be extended in vivo. This could be beneficial to patient populations as maximal dose amounts and maximal dosing frequencies could be achieved as a result of maintaining in vivo IC₅₀ for longer periods of time.

In some embodiments, an antigen binding protein comprising a constant region may have reduced ADCC and/or complement activation or effector functionality. The constant domain may comprise a naturally disabled constant region of IgG2 or IgG4 isotype or a mutated IgG1 constant domain. Examples of suitable modifications are described in EP0307434. One way to achieve Fc disablement may comprise the substitutions of alanine residues at positions 235 and 237 (EU index numbering) of the heavy chain constant region, i.e. L235A and G237A (commonly referred to as “LAGA” mutations). Another example may comprise substitution with alanines at positions 234 and 235 (EU index numbering), i.e. L234A and L235A (commonly referred to as “LALA” mutations). In some embodiments, the Fc effector function of an antigen binding protein disclosed herein may be disabled using the LAGA mutation.

Additional alterations and mutations to decrease effector function may include: (with reference to IgG1 unless otherwise noted): a glycosylated N297A or N297Q or N297G; L235E; IgG4:F234A/L235A; and chimeric IgG2/IgG4. IgG2: H268Q/V309L/A330S/P331S, and IgG2: V234A/G237A/P238S/H268A/V309L/A330S/P331S can reduce FcγR and C1q binding (U.S. Pat. No. 8,961,967).

Other mutations that decrease effector function may include L234F/L235E/P331S; a chimeric antibody created using the CH1 and hinge region from human IgG2 and the CH2 and CH3 regions from human IgG4; IgG2m4, based on the IgG2 isotype with four key amino acid residue changes derived from IgG4 (H268Q, V309L, A330S and P331S); IgG26 which contains V234A/G237A/P238S/H268A/V309L/A330S/P331S substitutions to eliminate affinity for Fcγ receptors and C1q complement protein; IgG2m4 (H268Q/V309L/A330S/P331S, changes to IgG4); IgG4 (S228P/L234A/L235A); huIgG1 L234A/L235A (AA); huIgG4 S228P/L234A/L235A; IgG1σ (L234A/L235A/G237A/P238S/H268A/A330S/P331S); IgG4θ1 (S228P/F234A/L235A/G237A/P238S); and IgG4θ2 (S228P/F234A/L235A/G236/G237A/P238S).

In some embodiments, an antigen binding protein disclosed herein may comprise one or more modifications selected from a mutated constant domain such that the antibody has enhanced effector functions/ADCC and/or complement activation. Examples of suitable modifications are described in U.S. Pat. No. 6,737,056, WO2004063351 and WO2004029207. The antigen binding protein may comprise a constant domain with an altered glycosylation profile such that the antigen binding protein has enhanced effector functions/ADCC and/or complement activation. Examples of suitable methodologies to produce an antigen binding protein with an altered glycosylation profile are described in WO2003/011878, WO2006/014679 and EP1229125.

### Half-Life

Half-life refers to the time required for the serum concentration of an antigen binding protein to reach half of its original value. The serum half-life of proteins can be measured by pharmacokinetic studies according to the method described by Kim et al., 1994, Eur. J. of Immuno. 24: 542-548. According to this method, radio-labelled protein is injected intravenously into mice and its plasma concentration is periodically measured as a function of time, for example, at about 3 minutes to about 72 hours after the injection. Other methods for pharmacokinetic analysis and determination of the half-life of a molecule will be familiar to those skilled in the art.

Antigen binding proteins of the present disclosure may have amino acid modifications that increase the affinity of the constant domain or fragment thereof for FcRn. Increasing the half-life (i.e., serum half-life) of therapeutic and diagnostic IgG antibodies and other bioactive molecules has many benefits including reducing the amount and/or frequency of dosing of these molecules. In one embodiment, an antigen binding protein of the invention comprises all or a portion (an FcRn binding portion) of an IgG constant domain having one or more of the following amino acid modifications.

For example, with reference to IgG1, M252Y/S254T/T256E (commonly referred to as “YTE” mutations) and M428L/N434S (commonly referred to as “LS” mutations) increase FcRn binding at pH 6.0 (Wang et al. 2018).

Half-life can also be enhanced by T2500/M428L, V2591/V308F/M428L, N434A, and T307A/E380A/N434A mutations (with reference to IgG1 and Kabat numbering) (Monnet et al.).

Half-life and FcRn binding can also be extended by introducing H433K and N434F mutations (commonly referred to as “HN” or “NHance” mutations) (with reference to IgG1) (WO2006/130834).

WO00/42072 discloses a polypeptide comprising a variant Fc region with altered FcRn binding affinity, which polypeptide comprises an amino acid modification at any one or more of amino acid positions 238, 252, 253, 254, 255, 256, 265, 272, 286, 288, 303, 305, 307, 309, 311, 312, 317, 340, 356, 360, 362, 376, 378, 380, 386,388, 400, 413, 415, 424, 433, 434, 435, 436, 439, and 447 of the Fc region (EU index numbering).

WO02/060919 discloses a modified IgG comprising an IgG constant domain comprising one or more amino acid modifications relative to a wild-type IgG constant domain, wherein the modified IgG has an increased half-life compared to the half-life of an IgG having the wild-type IgG constant domain, and wherein the one or more amino acid modifications are at one or more of positions 251, 253, 255, 285-290, 308-314, 385-389, and 428-435.

Shields et al. (2001, J Biol Chem; 276:6591-604) used alanine scanning mutagenesis to alter residues in the Fc region of a human IgG1 antibody and then assessed the binding to human FcRn. Positions that effectively abrogated binding to FcRn when changed to alanine include 1253, S254, H435, and Y436. Other positions showed a less pronounced reduction in binding as follows: E233-G236, R255, K288, L309, S415, and H433. Several amino acid positions exhibited an improvement in FcRn binding when changed to alanine; notable among these are P238, T256, E272, V305, T307, Q311, D312, K317, D376, E380, E382, S424, and N434. Many other amino acid positions exhibited a slight improvement (D265, N286, V303, K360, Q362, and A378) or no change (S239, K246, K248, D249, M252, E258, T260, S267, H268, S269, D270, K274, N276, Y278, D280, V282, E283, H285, T289, K290, R292, E293, E294, Q295, Y296, N297, S298, R301, N315, E318, K320, K322, S324, K326, A327, P329, P331, E333, K334, T335, S337, K338, K340, Q342, R344, E345, Q345, Q347, R356, M358, T359, K360, N361, Y373, S375, S383, N384, Q386, E388, N389, N390, K392, L398, S400, D401, K414, R416, Q418, Q419, N421, V422, E430, T437, K439, S440, S442, S444, and K447) in FcRn binding.

The most pronounced effect with respect to improved FcRn binding was found for combination variants. At pH 6.0, the E380A/N434A variant showed over 8-fold better binding to FcRn, relative to native IgG1, compared with 2-fold for E380A and 3.5-fold for N434A. Adding T307A to this resulted in a 12-fold improvement in binding relative to native IgG1. In one embodiment, the antigen binding protein of the invention comprises the E380A/N434A mutations and has increased binding to FcRn.

Dall'Acqua et al. (2002, J Immunol.; 169:5171-80) describes random mutagenesis and screening of human IgG1 hinge-Fc fragment phage display libraries against mouse FcRn. They disclosed random mutagenesis of positions 251, 252, 254-256, 308, 309, 311, 312, 314, 385-387, 389, 428, 433, 434, and 436. The major improvements in IgG1-human FcRn complex stability occur when substituting residues located in a band across the Fc-FcRn interface (M252, S254, T256, H433, N434, and Y436) and to lesser extent substitutions of residues at the periphery, such as V308, L309, Q311, G385, Q386, P387, and N389. The variant with the highest affinity to human FcRn was obtained by combining the M252Y/S254T/T256E (“YTE”) and H433K/N434F/Y436H mutations and exhibited a 57-fold increase in affinity relative to the wild-type IgG1. The in vivo behavior of such a mutated human IgG1 exhibited a nearly 4-fold increase in serum half-life in cynomolgus monkey as compared to wild-type IgG1.

The present disclosure therefore provides an antigen binding protein with optimized binding to FcRn. In some embodiments, the antigen binding protein comprises at least one amino acid modification in the Fc region of said antigen binding protein, wherein said modification is at an amino acid position selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region.

Additionally, various publications describe methods for obtaining physiologically active molecules with modified half-lives, either by introducing an FcRn-binding polypeptide into the molecules (WO97/43316, U.S. Pat. Nos. 5,869,046, 5,747,035, WO96/32478 and WO91/14438) or by fusing the molecules with antibodies whose FcRn-binding affinities are preserved, but affinities for other Fc receptors have been greatly reduced (WO99/43713), or fusing with FcRn binding domains of antibodies (WO00/09560, U.S. Pat. No. 4,703,039).

FcRn affinity enhanced Fc variants to improve both antibody cytotoxicity and half-life were identified in screens at pH 6.0. The selected IgG variants can be produced as low fucosylated molecules. The resulting variants show increased serum persistence in hFcRn mice, as well as conserved enhanced ADCC (Monnet et al.) Exemplary variants include (with reference to IgG1 and Kabat numbering): P230T/V303A/K322R/N389T/F404L/N434S; P228R/N434S; Q311R/K334R/Q342E/N434Y; C226G/Q386R/N434Y; T307P/N389T/N434Y; P230S/N434S; P230T/V305A/T307A/A378V/L398P/N434S; P230T/P387S/N434S; P230Q/E269D/N434S; N276S/A378V/N434S; T307A/N315D/A330V/382V/N389T/N434Y; T256N/A378V/S383N/N434Y; N315D/A330V/N361D/A387V/N434Y; V2591/N315D/M428L/N434Y; P230S/N315D/M428L/N434Y; F241L/V264E/T307P/A378V/H433R; T250A/N389K/N434Y; V305A/N315D/A330V/P395A/N434Y; V264E/Q386R/P396L/N434S/K439R; E294del/T307P/N434Y (wherein ‘del’ indicates a deletion).

### Antibody Drug Conjugate

Also provided is an immunoconjugate (interchangeably referred to as an “antibody-drug conjugate”, “ADC” or “antigen binding protein-drug conjugate”) comprising an antigen binding protein according to the disclosure conjugated to one or more drugs, such as a cytotoxic agent, such as a chemotherapeutic agent, an immunotherapeutic agent, a growth inhibitory agent, a toxin (e.g., a protein toxin, such as an enzymatically active toxin of bacterial, fungal, plant, or animal origin, or fragments thereof), an antiviral agent, a radioactive isotope (i.e., a radioconjugate), an antibiotic, or a small interfering RNA (siRNA).

Immunoconjugates have been used for the local delivery of cytotoxic agents, i.e., drugs that kill or inhibit the growth or proliferation of cells, in the treatment of cancer (Lambert, J. (2005) Curr. Opinion in Pharmacology 5:543-549; Wu et al. (2005) Nature Biotechnology 23(9):1137-1146; Payne, G. (2003) i 3:207-212; Syrigos and Epenetos (1999) Anticancer Research 19:605-614; Niculescu-Duvaz and Springer (1997) Adv. Drug Deliv. Rev. 26:151-172; U.S. Pat. No. 4,975,278). Immunoconjugates allow for, inter alia, the targeted delivery of a drug moiety to a tumor, and intracellular accumulation therein, where systemic administration of unconjugated drugs may result in unacceptable levels of toxicity to normal cells (Tsuchikama and An, Protein and Cell, (2018) 9: 33-46). Immunoconjugates can enable selective delivery of a potent cytotoxic payload to target cancer cells, resulting in improved efficacy, reduced systemic toxicity, and preferable pharmacokinetics (PK)/pharmacodynamics (PD) and biodistribution compared to traditional chemotherapy (Tsuchikama and An 2018); Beck A. et al (2017) Nature Rev. Drug Disc. 16: 315-337).

Both polyclonal antibodies and monoclonal antibodies have been reported as useful in these strategies (Rowland et al., (1986) Cancer Immunol. Immunother. 21:183-87). Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al (2000) J. Nat. Cancer Inst. 92(19):1573-1581; Mandler et al (2000) Bioorganic & Med. Chem. Letters 10:1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342).

In certain embodiments, an immunoconjugate comprises an antigen binding protein, such as an antibody, and a drug, such as toxin, such as a chemotherapeutic agent. The drug can be modified (e.g., via standard synthetic chemistry) to allow its chemical attachment (e.g., to contain a reaction handle to allow its chemical attachment) to a reactive end of a linker that joins the drug to the antigen binding protein.

### Drug Component of Immunoconjugate

Drugs, such as chemotherapeutic agents, useful in the generation of immunoconjugates are described herein. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. See, e.g., WO 93/21232 published Oct. 28, 1993.

In addition to toxins, a radioactive material, such as a radionucleotide, may be used as the drug in an ADC. A variety of radionucleotides are available for the production of radioconjugated antibodies. Examples include 212Bi, 1311, 1311n, 90Y, and 186Re.

Antigen binding proteins (such as antibodies) of the present disclosure may also be conjugated to one or more toxins, including, but not limited to, a calicheamicin, a maytansinoid, a dolastatin, an aurostatin, a trichothecene, and CC1065, and a derivative of these toxins that have toxin activity. Suitable cytotoxic agents include, but are not limited to, an auristatin including dovaline-valine-dolaisoleunine-dolaproine-phenylalanine (MMAF) and monomethyl auristatin E (MMAE) as well as an ester form of MMAE, a DNA minor groove binding agent, a DNA minor groove alkylating agent, an enediyne, a lexitropsin, a duocarmycin, a taxane (such as paclitaxel and docetaxel), a puromycin, a dolastatin, a maytansinoid, and a vinca alkaloid. Specific cytotoxic agents include topotecan, morpholino-doxorubicin, rhizoxin, cyanomorpholino-doxorubicin, dolastatin-10, echinomycin, combretatstatin, chalicheamicin, maytansine, DM-1, DM-4, and netropsin. Other suitable cytotoxic agents include anti-tubulin agents, such as an auristatin, a vinca alkaloid, a podophyllotoxin, a taxane, a baccatin derivative, a cryptophysin, a maytansinoid, a combretastatin, or a dolastatin. Antitubulin agents include dimethylvaline-valine-dolaisoleuine-dolaproine-phenylalanine-p-phenylened-iamine (AFP), MMAF, MMAE, auristatin E, vincristine, vinblastine, vindesine, vinorelbine, VP-16, camptothecin, paclitaxel, docetaxel, epothilone A, epothilone B, nocodazole, colchicines, colcimid, estramustine, cemadotin, discodermolide, maytansine, DM-1, DM-4, and eleutherobin.

Antibody drug conjugates can be produced by conjugating the anti-tubulin agent monomethylauristatin E (MMAE) or monomethylauristatin F (MMAF) to an antigen binding protein (such as an antibody). In the case of MMAE, the linker can consist of a thiol-reactive maleimide, a caproyl spacer, the dipeptide valine-citrulline, or p-aminobenzyloxycarbonyl, a self-immolative fragmenting group. In the case of MMAF, a protease-resistant maleimidocaproyl linker can be used. The conjugation process leads to heterogeneity in drug-antibody attachment, varying in both the number of drugs bound to each antibody molecule (mole ratio [MR]), and the site of attachment. The most prevalent species is the material with an MR=4; less prevalent are materials with MR of 0, 2, 6, and 8. The overall average drug-to-antibody MR is approximately 4.

### Auristatins and Dolastatins

In some embodiments, the immunoconjugate comprises an antigen binding protein (such as an antibody) conjugated to a dolastatin or a dolostatin peptidic analog or derivative, an auristatin (U.S. Pat. Nos. 5,635,483; 5,780,588). Dolastatins and auristatins have been shown to interfere with microtubule dynamics, GTP hydrolysis, and nuclear and cellular division (Woyke et al. (2001) Antimicrob. Agents and Chemother. 45(12):3580-3584) and have anticancer (U.S. Pat. No. 5,663,149) and antifungal activity (Pettit et al. (1998) Antimicrob. Agents Chemother. 42:2961-2965). The dolastatin or auristatin (a pentapeptide derivative of dolastatin) drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF, disclosed in “Monomethylvaline Compounds Capable of Conjugation to Ligands,” U.S. Pat. No. 7,498,298. As used herein, the abbreviation “MMAE” refers to monomethyl auristatin E. As used herein the abbreviation “MMAF” refers to dovaline-valine-dolaisoleuine-dolaproine-phenylalanine.

Typically, peptide-based drug moieties can be prepared by forming a peptide bond between two or more amino acids and/or peptide fragments. Such peptide bonds can be prepared, for example, according to the liquid phase synthesis method (see E. Schroder and K. Lubke, “The Peptides,” volume 1, pp 76-136, 1965, Academic Press) that is well known in the field of peptide chemistry. The auristatin/dolastatin drug moieties may be prepared according to the methods of: U.S. Pat. Nos. 5,635,483; 5,780,588; Pettit et al. (1989) J. Am. Chem. Soc. 111:5463-5465; Pettit et al. (1998) Anti-Cancer Drug Design 13:243-277; Pettit, G. R., et al. Synthesis, 1996, 719-725; and Pettit et al. (1996) J. Chem. Soc. Perkin Trans. 15:859-863. See also Doronina (2003) Nat Biotechnol 21(7):778-784; “Monomethylvaline Compounds Capable of Conjugation to Ligands,” U.S. Pat. No. 7,498,298, (disclosing, e.g., linkers and methods of preparing monomethylvaline compounds such as MMAE and MMAF conjugated to linkers). Biologically active organic compounds that act as cytotoxic agents, specifically pentapeptides, are disclosed in U.S. Pat. Nos. 6,884,869; 7,498,298; 7,098,308; 7,256,257; and 7,423,116.

### Maytansine and Maytansinoids

Maytansinoids are mitototic inhibitors that act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Highly cytotoxic maytansinoid drugs can be prepared from ansamitocin precursors produced by fermentation of microorganisms such as Actinosynnema. Methods for isolating ansamitocins are described in U.S. Pat. No. 6,573,074. Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Antibody-maytansinoid conjugates are prepared by chemically linking an antigen binding protein (such as an antibody) to a maytansinoid molecule without significantly diminishing the biological activity of either the antibody or the maytansinoid molecule. See, e.g., U.S. Pat. No. 5,208,020. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Maytansinoids are maytansinol and maytansinol analogues modified in the aromatic ring or at other positions of the maytansinol molecule, such as various maytansinol esters. Methods for preparing maytansinoids for linkage with antibodies are disclosed, e.g., in U.S. Pat. Nos. 6,570,024 and 6,884,874.

### Calicheamicin

The calicheamicin family of antibiotics is capable of producing double-stranded DNA breaks at sub-picomolar concentrations. For the preparation of conjugates of the calicheamicin family, see, e.g., U.S. Pat. Nos. 5,712,374, 5,714,586, 5,739,116, 5,767,285, 5,770,701, 5,770,710, 5,773,001, and 5,877,296. Structural analogues of calicheamicin that may be used include, but are not limited to, γ1I, α2I, α3I, N-acetyl γ1I, PSAG and τI1 (Hinman et al., Cancer Research 53:3336-3342 (1993), Lode et al., Cancer Research 58:2925-2928 (1998) and the aforementioned U.S. patents). Another anti-tumor drug that the antibody can be conjugated to is QFA, which is an antifolate. Both calicheamicin and QFA have intracellular sites of action and do not readily cross the plasma membrane. Therefore, cellular uptake of these agents through antibody mediated internalization greatly enhances their cytotoxic effects.

### Other Cytotoxic Agents

Other cytotoxic agents, such as antitumor agents, that can be conjugated to an antigen binding protein (such as an antibody) include BCNU, streptozoicin, vincristine and 5-fluorouracil, the family of agents known collectively LL-E33288 complex described in U.S. Pat. Nos. 5,053,394 and 5,770,710, as well as esperamicins (U.S. Pat. No. 5,877,296).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antigen binding protein (such as an antibody) and a compound with nucleolytic activity (e.g., a ribonuclease or a DNA endonuclease such as a deoxyribonuclease; DNase).

For selective destruction of the tumor, the antigen binding protein (such as an antibody) may comprise a highly radioactive atom. A variety of radioactive isotopes are available for the production of radioconjugated antibodies. Examples include At211, I131, I125, Y90, Re186, Re188, Sm153, Bi212, P32, Pb212 and radioactive isotopes of Lu. When the conjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc99m or 1123, Re186, Re188 and In111 can be attached via a cysteine residue in the peptide. Yttrium-90 can be attached via a lysine residue. The IODOGEN method (Fraker et al. (1978) Biochem. Biophys. Res. Commun. 80: 49-57) can be used to incorporate iodine-123. “Monoclonal Antibodies in Immunoscintigraphy” (Chatal, CRC Press 1989) describes other methods in detail.

In some cases, an anti-BCMA antigen binding protein disclosed herein is an immunoconjugate comprising an antigen binding protein as herein described including, but not limited to, an antibody conjugated to one or more cytotoxic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate). In some cases, the anti-BCMA antigen binding protein can be conjugated to a toxin such as an auristatin, e.g., monomethyl auristatin E (MMAE) or monomethyl auristatin F (MMAF). In some embodiments, the anti-BCMA antigen binding protein is conjugated to AFP, MMAF, MMAE, AEB, AEVB or auristatin E. In some embodiments, the anti-BCMA antigen binding protein is conjugated to paclitaxel, docetaxel, CC-1065, SN-38, topotecan, morpholino-doxorubicin, rhizoxin, cyanomorpholino-doxorubicin, dolastatin-10, echinomycin, combretatstatin, calicheamicin, or netropsin. In some embodiments, the anti-BCMA antigen binding protein is conjugated to an auristatin, a maytansinoid, or calicheamicin. In some embodiments, the anti-BCMA antigen binding protein is conjugated to AFP, MMAP, MMAE, AEB, AEVB, auristatin E, vincristine, vinblastine, vindesine, vinorelbine, VP-16, camptothecin, paclitaxel, docetaxel, epothilone A, epothilone B, nocodazole, colchicines, colcimid, estramustine, cemadotin, discodermolide, maytansinol, maytansine, DM1, DM2, DM3, DM4 or eleutherobin.

In some cases, an anti-BCMA antigen binding protein conjugated to a toxin can include a heavy chain having a V_{H} CDR1 comprising, consisting essentially of, or consisting of the amino acid sequence set forth in SEQ ID NO:1, a V_{H} CDR2 comprising, consisting essentially of, or consisting of the amino acid sequence set forth in SEQ ID NO:2, and a V_{H} CDR3 comprising, consisting essentially of, or consisting of the amino acid sequence set forth in SEQ ID NO:3. For example, an anti-BCMA antigen binding protein conjugated to a toxin described herein can include a heavy chain variable region including the amino acid sequence set forth in SEQ ID NO:7. In some cases, an anti-BCMA antigen binding protein conjugated to a toxin described herein can include a heavy chain comprising the amino acid sequence set forth in SEQ ID NO:9.

In some cases, an anti-BCMA antigen binding protein conjugated to a toxin can include a light chain having a V_{L} CDR1 comprising, consisting essentially of, or consisting of the amino acid sequence set forth in SEQ ID NO:4, a V_{L} CDR2 comprising, consisting essentially of, or consisting of the amino acid sequence set forth in SEQ ID NO:5, and a V_{L} CDR3 comprising, consisting essentially of, or consisting of the amino acid sequence set forth in SEQ ID NO:6. An anti-BCMA antigen binding protein conjugated to a toxin described herein can include a light chain variable region including the amino acid sequence set forth in SEQ ID NO:8. In some cases, an anti-BCMA antigen binding protein conjugated to a toxin described herein can include a light chain comprising the amino acid sequence set forth in SEQ ID NO: 10.

In some cases, an anti-BCMA antigen binding protein conjugated to a toxin can include a heavy chain having a V_{H} CDR1 including the amino acid sequence set forth in SEQ ID NO:1, a V_{H} CDR2 including the amino acid sequence set forth in SEQ ID NO:2, and a V_{H} CDR3 including the amino acid sequence set forth in SEQ ID NO:3, and can include a light chain having a V_{L} CDR1 including the amino acid sequence set forth in SEQ ID NO:4, a V_{L} CDR2 including the amino acid sequence set forth in SEQ ID NO:5, and a V_{L} CDR3 including the amino acid sequence set forth in SEQ ID NO:6. For example, an anti-BCMA antigen binding protein conjugated to a toxin can include a heavy chain variable region including the amino acid sequence set forth in SEQ ID NO:7 and can include a light chain variable region including the amino acid sequence set forth in SEQ ID NO:8. In some cases an anti-BCMA antigen binding protein conjugated to a toxin described herein can include a heavy chain comprising the amino acid sequence set forth in SEQ ID NO:9 and can include a light chain comprising the amino acid sequence set forth in SEQ ID NO: 10.

In some embodiments, an anti-BCMA antigen binding protein disclosed herein comprises the heavy chain variable region of SEQ ID NO: 11, 15 or 19. In some embodiments, an anti-BCMA antigen binding protein disclosed herein comprises the light chain variable region of SEQ ID NO: 12, 16 or 20. In some embodiments, an anti-BCMA antigen binding protein disclosed herein comprises the heavy chain region of SEQ ID NO: 13, 17, 22, or 24. In some embodiments, an anti-BCMA antigen binding protein disclosed herein comprises the light chain region of SEQ ID NO: 14, 18, 23 or 25. In some embodiments, an anti-BCMA antigen binding protein disclosed herein comprises the heavy chain variable region of SEQ ID NO: 11 and the light chain variable region of SEQ ID NO: 12, the heavy chain variable region of SEQ ID NO: 15 and the light chain variable region of SEQ ID NO: 16, or the heavy chain variable region of SEQ ID NO: 19 and the light chain variable region of SEQ ID NO: 20. In some embodiments, an anti-BCMA antigen binding protein disclosed herein comprises the heavy chain region of SEQ ID NO: 13 and the light chain of SEQ ID NO: 14, the heavy chain region of SEQ ID NO: 17 and the light chain of SEQ ID NO: 18, the heavy chain region of SEQ ID NO: 22 and the light chain of SEQ ID NO: 23 or the heavy chain region of SEQ ID NO: 24 and the light chain of SEQ ID NO: 25. In some embodiments, an anti-BCMA antigen binding protein disclosed herein is an scFV-fc comprising SEQ ID NO: 21.

In some cases, the anti-BCMA antigen binding protein can be an immunoconjugate having the following general structure:
ABP-((Linker)n-Ctx)m
wherein
ABP is an antigen binding protein
Linker is either absent or any a cleavable or non-cleavable linker
Ctx is any cytotoxic agent described herein
n is 0, 1, 2, or 3 and
m is 1, 2, 3, 4, 5, 6, 7, 8, 9 or 10.

Exemplary linkers may include 6-maleimidocaproyl (MC), maleimidopropanoyl (MP), valine-citrulline (val-cit), alanine-phenylalanine (ala-phe), p-aminobenzyloxycarbonyl (PAB), N-Succinimidyl 4-(2-pyridylthio)pentanoate (SPP), N-succinimidyl 4-(N-maleimidomethyl)cyclohexane-1 carboxylate (SMCC), and N-succinimidyl (4-iodo-acetyl) aminobenzoate (SIAB).

In some cases, the anti-BCMA antigen binding protein can be an immunoconjugate containing a monoclonal antibody linked to MMAE or MMAF. In another embodiment, the anti-BCMA antigen binding protein can be an immunoconjugate containing a monoclonal antibody linked to MMAE or MMAF by an MC linker as depicted in the following structures:

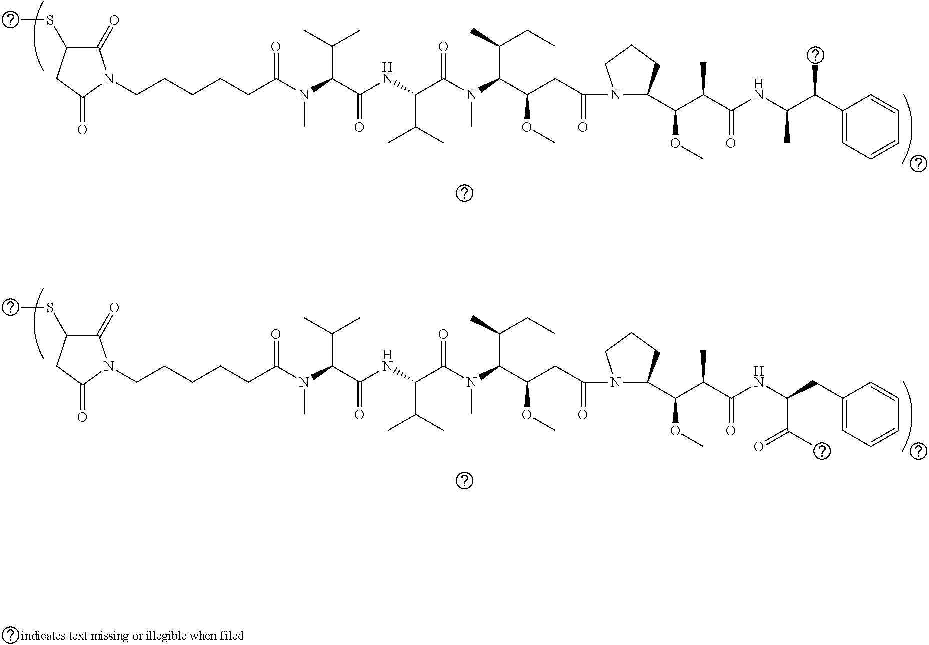 [SMILES: CC[C@H](C)[C@@H]([C@@H](CC(=O)N1CCC[C@H]1[C@H](OC)[C@@H](C)C(=O)N[C@H](C)[C@@H](C)c1ccccc1)OC)N(C)C(=O)[C@@H](NC(=O)[C@H](C(C)C)N(C)C(=O)CCCCCN1C(=O)CC(SC)C1=O)C(C)C ; CC[C@H](C)[C@@H]([C@@H](CC(=O)N1CCC[C@H]1[C@H](OC)[C@@H](C)C(=O)N[C@@H](Cc1ccccc1)C(C)=O)OC)N(C)C(=O)[C@@H](NC(=O)[C@H](C(C)C)N(C)C(=O)CCCCCN1C(=O)CC(SC)C1=O)C(C)C]

In some cases, the anti-BCMA antigen binding protein can be the antibody belantamab. In another embodiment, the anti-BCMA antigen binding protein can be the immunoconjugate belantamab mafodotin.

In some cases, the conjugated antibodies (antibody-drug conjugates or ADCs) of the present disclosure can be powerful anti-cancer agents designed to allow specific targeting of highly potent cytotoxic agents to tumor cells while sparing healthy tissues. Despite the use of tumor-specific antibodies, the emerging clinical data with ADCs indicates that adverse events frequently occur before ADCs have reached their optimal therapeutic dose. As such, despite these ADCs being highly active in preclinical tumor models their therapeutic window in the clinic is narrow and dosing regimens seem hampered by dose-limiting toxicities that could not always be predicted based on data from preclinical models.

In some cases, therapies which could be combined to synergistically enhance therapeutic efficacy without worsening the safety profile can be a major advancement in the treatment of cancer patients particularly with regards to the incidence and severity of treatment-emergent adverse events such as ocular toxicity.

In some cases, a combination with a drug which could enhance the efficacy of doses leading to overall responses rates (ORR) which are markedly higher whilst having the best benefit-risk profile can lead to a paradigm shift in the management of patients treated with such antigen binding proteins.

### Disorders

In some cases, a combination disclosed herein can treat a B-cell disorder. B-cell disorders can be divided into defects of B-cell development/immunoglobulin production (immunodeficiencies) and excessive/uncontrolled proliferation (lymphomas, leukemias). As used herein, B-cell disorder refers to both types of diseases, and methods are provided for treating B-cell disorders with an antigen binding protein.

Examples of cancers and in particular B-cell mediated or plasma cell mediated diseases or antibody mediated diseases or disorders can include Multiple Myeloma (MM), chronic lymphocytic leukemia (CLL), Follicular Lymphoma (FL), Diffuse Large B-Cell Lymphoma (DLBCL), Non-secretory multiple myeloma, Smoldering multiple myeloma, Monoclonal gammopathy of undetermined significance (MGUS), Solitary plasmacytoma (Bone, Extramedullary), Lymphoplasmacytic lymphoma (LPL), Waldenström's Macroglobulinemia, Plasma cell leukemia, Primary Amyloidosis (AL), Heavy chain disease, Systemic lupus erythematosus (SLE), POEMS syndrome/osteosclerotic myeloma, Type I and II cryoglobulinemia, Light chain deposition disease, Goodpasture's syndrome, Idiopathic thrombocytopenic purpura (ITP), Acute glomerulonephritis, Pemphigus and Pemphigoid disorders, and Epidermolysis bullosa acquisita; or any Non-Hodgkin's Lymphoma B-cell leukemia (NHL) and Hodgkin's lymphoma (HL). In some cases, the disease or disorder can be selected from the group consisting of Multiple Myeloma (MM), Non-Hodgkin's Lymphoma B-cell leukemia (NHL), Follicular Lymphoma (FL), and Diffuse Large B-Cell Lymphoma (DLBCL). In some cases, the disease can be Multiple Myeloma or Non-Hodgkin's Lymphoma B-cell leukemia (NHL). In some cases, the disease can be Multiple Myeloma.

In some cases, the cancer may be a hematopoietic (or hematologic or haematological or blood-related) cancer, for example, cancers derived from blood cells or immune cells, which may be referred to as “liquid tumors”. In some cases, the cancer can be a B-cell related cancer and particularly a BCMA-expressing cancer. In some cases, the cancer can be a leukemia such as chronic myelocytic leukemia, acute myelocytic leukemia, chronic lymphocytic leukemia and acute lymphocytic leukemia. In another case, the cancer can be a lymphoma such as non-Hodgkin's lymphoma, Hodgkin's lymphoma; and the like. In another case, the cancer can be a plasma cell malignancy such as multiple myeloma, and Waldenstrom's macroglobulinemia. In some embodiments, a combination disclosed herein treats AL amyloidosis.

In some cases, the cancer can be multiple myeloma. In some cases, the cancer can be relapsed and/or refractory multiple myeloma. In some cases, the patient with relapsed and/or refractory multiple myeloma has been previously treated with at least one, at least two, at least three or at least four therapeutics to treat the multiple myeloma.

### Prior Treatment

In some cases, a subject described herein may have had 0, 1, 2, 3, or 4 or more prior lines of treatment before being treated with the combinations described herein. In another embodiment, the subject may have relapsed and/or refractory multiple myeloma and have had 0, 1, 2, 3, or 4 or more prior lines of treatment before being treated with the combinations described herein. In another embodiment, the subject has been previously treated with at least 3 prior lines that may include the following: an immunomodulatory drug (IMiD), a proteasome inhibitor (PI) and anti-CD38 treatment (e.g., daratumumab) or combinations thereof. Lines of therapy may be defined by consensus panel of the International Myeloma Workshop (IMWG).

In some cases, a subject may have had 0, 1, 2, 3, or 4 or more prior lines of treatment before being treated with the combinations described herein, wherein the one or more of the prior lines of treatment were unsuccessful. In some cases, adverse events connected to the prior line of treatment forced discontinuation of the prior line of treatment. When a mammal (e.g., a human) that can be treated as described herein is a mammal that has had 0, 1, 2, 3, or 4 or more prior lines of treatment before being treated as described herein, the prior treatment can be any appropriate treatment. For example, a mammal that has had 0, 1, 2, 3, or 4 or more prior lines of treatment before being treated as described herein, can have been previously treated with an immunomodulatory drug (IMiD), a proteasome inhibitor (PI), an anti-CD38 treatment or combinations thereof.

In some cases, a subject that had prior lines of treatment may have a cancer that is recurrent, relapsed, and/or refractory. In some cases, a cancer can be a primary cancer. In some cases, a cancer can be a metastatic cancer. In some cases, a cancer can be a chemo-resistant cancer. In some cases, a cancer can be a B cell cancer (e.g., leukemias and lymphomas). Examples of cancers that can be treated as described herein include, without limitation, multiple myeloma (MM), chronic lymphocytic leukemia (CLL), chronic myelocytic leukemia, acute myelocytic leukemia, acute lymphocytic leukemia, follicular lymphoma (FL), diffuse large B-cell lymphoma (DLBCL), non-secretory multiple myeloma, smoldering multiple myeloma, monoclonal gammopathy of undetermined significance (MGUS), solitary plasmacytoma (e.g., solitary plasmacytoma of the bone and extramedullary solitary plasmacytoma), lymphoplasmacytic lymphoma (LPL), Waldenström's macroglobulinemia, plasma cell leukemia, primary amyloidosis (AL), heavy chain disease, systemic lupus erythematosus (SLE), POEMS syndrome, osteosclerotic myeloma, Type I and II cryoglobulinemia, light chain deposition disease, Goodpasture's syndrome, idiopathic thrombocytopenic purpura (ITP), acute glomerulonephritis, pemphigus and pemphigoid disorders, epidermolysis bullosa acquisita, non-Hodgkin's lymphomas, B-cell leukaemia, and Hodgkin's lymphomas.

### Statement of Use

In some cases, a combination disclosed herein can be used to treat a disease or condition for which a BCMA antigen binding protein is indicated, for example a cancer. Such a treatment may comprise: (i) an anti-BCMA antigen binding protein or an ADC having binding specificity for a BCMA polypeptide and (ii) one or more CELMODs. In some cases, a mammal (e.g., a human such as a human having cancer) can be administered: (i) anti-BCMA antigen binding protein or an ADC having binding specificity for a BCMA polypeptide and (ii) one or more CELMODs. In some cases, a combination disclosed herein targets the cytotoxic agent of the ADC to cells (e.g., cancer cells) expressing a BCMA polypeptide (e.g., expressing a BCMA polypeptide on the cell surface) and to stimulate (e.g., induce or enhance) an immune response against cells (e.g., cancer cells) expressing a cancer related antigen. In some cases, a BCMA antigen binding protein and a CELMOD can be bound to the same cancer cell. In some cases, an anti-BCMA antigen binding protein and a CELMOD can be bound to different cancer cells. In some cases, an anti-BCMA antigen binding protein and a CELMOD can interact with the same or different cancer cells.

In some cases, combinations disclosed herein may be for the treatment of a subject. The terms “individual”, “subject” and “patient” are used herein interchangeably. The subject can be a human. The subject may also be a mammal, such as a mouse, rat or primate (e.g., a marmoset or monkey). The subject can be a non-human animal. The subject to be treated may be a farm animal for example, a cow or bull, sheep, pig, ox, goat or horse or may be a domestic animal such as a dog or cat. The animal may be any age, or a mature adult animal. In some embodiments, treatment may be therapeutic, prophylactic or preventative. The subject may be one who is in need thereof. Those in need of treatment may include individuals already suffering from a medical disease in addition to those who may develop the disease in the future.

Thus, the compositions described herein can be used for prophylactic or preventative treatment. In this case, the compositions described herein can be administered to an individual in order to prevent or delay the onset of one or more aspects or symptoms of a disease. The subject can be asymptomatic. The subject may have a genetic predisposition to the disease. In some embodiments, a prophylactically effective amount of a combination disclosed herein can be administered to such an individual. In some embodiments, a prophylactically effective amount can be an amount which prevents or delays the onset of one or more aspects or symptoms of a disease described herein.

A combination disclosed herein may also be used in methods of therapy. The term “therapy” encompasses alleviation, reduction, or prevention of at least one aspect or symptom of a disease. For example, a combination disclosed herein may be used to ameliorate or reduce one or more aspects or symptoms of a disease described herein.

In some cases, a combination described herein may be used in an effective amount for therapeutic, prophylactic or preventative treatment. In some cases, a therapeutically effective amount of a combination described herein can be an amount effective to ameliorate or reduce one or more aspects or symptoms of the disease. In some cases, a combination disclosed herein may also have a generally beneficial effect on the subject's health, for example it can increase the subject's expected longevity.

In some cases, a combination described herein may not need to affect a complete cure or eradicate every symptom or manifestation of the disease to constitute a viable therapeutic treatment. As is recognized in the pertinent field, drugs employed as therapeutic agents may reduce the severity of a given disease state but need not abolish every manifestation of the disease to be regarded as useful therapeutic agents. Similarly, a prophylactically administered treatment need not be completely effective in preventing the onset of a disease in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur (for example by delaying the onset of the disease) or worsen in a subject, may be sufficient.

In some cases, the materials and methods provided herein can be used to reduce or eliminate the number of cancer cells present within a mammal (e.g., a human) having cancer. For example, a mammal in need thereof (e.g., a mammal having cancer) can be administered an anti-BCMA antigen binding protein and a CELMOD to reduce the number of cancer cells present within a mammal having cancer (e.g., the number of cancer cells present in a sample obtained from a mammal having cancer) by, for example, at least about 10, 20, 30, 40, 50, 60, 70, 80, 90, 95, or more percent. In some cases, there may be no cancer cells present within a sample obtained from a mammal having cancer. For example, a mammal in need thereof (e.g., a mammal having cancer) can be administered an anti-BCMA antigen binding protein and a CELMOD to reduce the size (e.g., volume) of one or more tumors present within a mammal having cancer by, for example, at least about 10, 20, 30, 40, 50, 60, 70, 80, 90, 95, or more percent. In some cases, the number of cancer cells present within a mammal being treated can be monitored. Any appropriate method can be used to determine whether the number of cancer cells present within a mammal is reduced. For example, imaging techniques can be used to assess the number of cancer cells present within a mammal.

In some cases, the materials and methods provided herein can be used to improve survival of a mammal (e.g., a human) having cancer. For example, a mammal in need thereof (e.g., a mammal having cancer) can be administered an anti-BCMA antigen binding protein and a CELMOD to improve survival of the mammal. For example, the materials and methods described herein can be used to improve the survival of a mammal having cancer by, for example, by at least about 10, 20, 30, 40, 50, 60, 70, 80, 90, 95, or more percent. For example, the materials and methods described herein can be used to improve the survival of a mammal having cancer by, for example, at least about 3 months (e.g., at least about 3 months, at least about 6 months, at least about 8 months, at least about 10 months, at least about 1 year, at least about 1.5 years, at least about 2 years, at least about 2.5 years, at least about 3 years, at least about 4 years, at least about 5 years, or more).

In some cases, the materials and methods provided herein can be used to treat of mammal (e.g., a human) having cancer such that the mammal can experience minimal, reduced, or no side effects. For example, when administered a combination disclosed herein (for example an anti-BCMA antigen binding protein and a CELMOD) the mammal can experience minimal, reduced, or no side effects as compared to a mammal having cancer and is administered the anti-BCMA antigen binding protein alone or the CELMOD alone. Examples of side effects that can be experienced by a mammal having cancer includes without limitation, one or more side effects selected from vision or eye changes such as findings on eye exam (keratopathy), decreased vision or blurred vision, nausea, low blood cell counts, fever, infusion-related reactions, tiredness, changes in kidney or liver function blood tests, thrombocytopenia, ocular toxicity (e.g., changes in corneal epithelium, dry eyes, irritation, redness, blurred vision, dry eyes, photophobia, and changes in visual acuity).

In some cases, provided is a combination comprising an anti-BCMA antigen binding protein and a CELMOD for use in preventing and/or reducing ocular toxicity in a patient with cancers, such as multiple myeloma. In one embodiment, ocular toxicity can be prevented or reduced when compared to a patient treated with the anti-BCMA antigen binding protein alone (monotherapy). In some embodiments, a combination disclosed herein, for example an anti-BCMA antigen binding protein and a CELMOD, decreases/slows the decline from baseline of 1 line on Snellen Visual Acuity as compared to treatment with the anti-BCMA antigen binding protein alone. In some embodiments, a combination disclosed herein, for example an anti-BCMA antigen binding protein and a CELMOD, decreases/slows the decline from baseline of 2 or 3 lines on Snellen Visual Acuity as compared to treatment with the anti-BCMA antigen binding protein alone. In some embodiments, a combination disclosed herein, for example an anti-BCMA antigen binding protein and a CELMOD, decreases/slows the decline from baseline by more than 3 lines on Snellen Visual Acuity as compared to treatment with the anti-BCMA antigen binding protein alone. In some embodiments, a combination disclosed herein, for example an anti-BCMA antigen binding protein and a CELMOD, decreases/slows the change from baseline on Snellen Visual Acuity as compared to treatment with the anti-BCMA antigen binding protein alone. In some embodiments, a combination disclosed herein, for example an anti-BCMA antigen binding protein and a CELMOD, decreases/slows the decrease in log MAR (logarithm of the minimum angle of resolution) units from baseline as compared to treatment with the anti-BCMA antigen binding protein alone. In some embodiments, a combination disclosed herein, for example an anti-BCMA antigen binding protein and a CELMOD, decreases/slows or prevents the progression of mild superficial keratopathy, moderate superficial keratopathy, severe superficial keratopathy or corneal epithelial defect in a subject as compared to treatment with the anti-BCMA antigen binding protein alone. In some embodiments, a combination disclosed herein, for example an anti-BCMA antigen binding protein and a CELMOD, prevents mild superficial keratopathy, moderate superficial keratopathy, severe superficial keratopathy or corneal epithelial defect in a subject as compared to treatment with the anti-BCMA antigen binding protein alone.

“Ocular toxicity” refers to any unintended exposure of a therapeutic agent to ocular tissue. Ocular toxicity can include changes in corneal epithelium, dry eyes, irritation, redness, blurred vision, dry eyes, photophobia, and/or changes in visual acuity.

Ophthalmic examination may be conducted by an ophthalmologist or optometrist. An ophthalmic examination may include one or more of the following:
1. Best corrected visual acuity,
2. Documentation of manifest refraction and the method used to obtain best corrected visual acuity,
3. Current glasses prescription (if applicable),
4. Intraocular pressure measurement,
5. Anterior segment (slit lamp) examination including fluorescein staining of the cornea and lens examination,
6. Dilated funduscopic examination, and/or
7. An ocular surface disease index (OSDI) which is visual function questionnaire that assess the impact of potential ocular change in vision on function and health-related quality of life.

The ophthalmic examination may occur before, during, and/or after treatment.

In one aspect, there is provided a method of treating cancer in a subject in need thereof comprising administering a therapeutically effective dose of an anti-BCMA antigen binding protein and a CELMOD according to the present disclosure.

### CELMOD

Disclosed herein is a combination that can comprise (i) an anti-BCMA antigen binding protein or an ADC having binding specificity for a BCMA polypeptide and (ii) one or more cereblon E3 ligase modulators (CELMODs). Any appropriate CELMOD(s) can be used to treat a mammal (e.g., a human) having cancer as described herein. In some instances, a CELMOD can have molecular weight less than 1000 Dalton. In some instances, a CELMOD can degrade an Ikaros protein, an Aiolos protein, or a combination thereof. In some instances, a CELMOD can have an IC₅₀ value less than 1 pM to cereblon (e.g., cereblon E3 ligase). In some instances, a CELMOD can be mezigdomide (CC-92480), iberdomide (CC-220), avadomide (CC-122), CC-90009, CC-99282, a pharmaceutically acceptable salt of any of the foregoing, or any combination thereof. In some instances, a CELMOD can be CC-99282 or a pharmaceutically acceptable salt thereof (e.g., administered orally and/or once daily). In some embodiments, a CELMOD disclosed herein is administered to the subject at least once daily, weekly, bi-weekly, every three weeks or monthly.

In some instances, a CELMOD can degrade GSPT1, for example being CC-90009 or a pharmaceutically acceptable salt thereof. In some embodiments, the CELMOD can be administered by an injection such as intravenously, and/or at least about 0.6 mg per unit dose (e.g., about: 0.6-1 mg, 0.6-2 mg, 0.6-3 mg, 0.6-4 mg, 0.6-5 mg, 1-2 mg, 1-3 mg, 1-4 mg, 1-5 mg, 2-3 mg, 2-4 mg, 2-5 mg, 3-4 mg, 3-5 mg, or 4-5 mg).

In some instances, a CELMOD can be mezigdomide (CC-92480) or a pharmaceutically acceptable salt thereof, e.g., administered orally. In some embodiments, the CELMOD can be present at about 1-5 mg, e.g., at about: 1-1.6, 1.6-2, 1.6-3, 1.6-4, 1.6-5, 1-2, 1-3, 1-4, 1-5, 2-3, 2-4, 2-5, 3-4, 3-5, or 4-5 mg, in the combination and/or administered once daily. In some embodiments, the CELMOD can be present in at least about: 0.1 mg to 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, or 1.6 mg in the combination. In some embodiments, the chemical name of the CELMOD can be Benzonitrile, 4-[4-[[4-[[[2-[(3S)-2,6-dioxo-3-piperidinyl]-2,3-dihydro-1-oxo-1H-isoindol-4-yl]oxy]methyl]phenyl]methyl]-1-piperazinyl]-3-fluoro. In some embodiments, the structural formula of the CELMOD can be

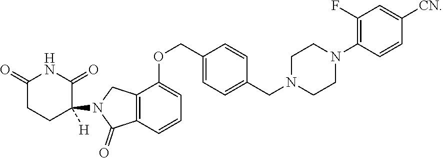 [SMILES: N#Cc1ccc(N2CCN(Cc3ccc(COc4cccc5c4CN([C@H]4CCC(=O)NC4=O)C5=O)cc3)CC2)c(F)c1]

In some instances, a CELMOD can be iberdomide (CC-220) or a pharmaceutically acceptable salt thereof such as iberdomide hydrochloride. In some embodiments, the CELMOD can be administered orally and/or at least about 0.6 or 1 mg daily. In some embodiments, the CELMOD can be present in at least about: 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, or 1.3 mg in the combination. In some embodiments, the CELMOD can be in a unit dose of about: 0.3-1 mg, 0.3-2 mg, 1-2 mg, 1-3 mg, 1-4 mg, 1-5 mg, 1-10 mg, 2-3 mg, 2-4 mg, 2-5 mg, 2-6 mg, 2-7 mg, 2-8 mg, 2-9 mg, 2-10 mg, 3-4 mg, 3-5 mg, 3-6 mg, 3-7 mg, 3-8 mg, 3-9 mg, 3-10 mg, 4-5 mg, 6-7 mg, 8-9 mg, 9-10 mg, 6-8 mg, 6-9 mg, or 6-10 mg. In some embodiments, the chemical name of the CELMOD can be S)-3-[4-({4-[(morpholin-4-yl)methyl]phenyl}methoxy)-1-oxo-1,3-dihydro-2H-isoindol-2-yl]piperidine-2,6-dione, or be 2,6-Piperidinedione,3-[1,3-dihydro-4-[[4-(4-morpholinylmethyl)phenyl]methoxy]-1-oxo-2H-isoindol-2-yl]-, (3S)—. In some embodiments, the structural formula of the CELMOD can be

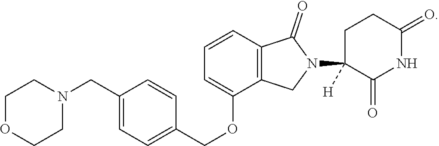 [SMILES: O=C1CC[C@H](N2Cc3c(OCc4ccc(CN5CCOCC5)cc4)cccc3C2=O)C(=O)N1]

In some instances, a CELMOD can be avadomide (CC-122) or a pharmaceutically acceptable salt thereof (e.g., avadomide hydrochloride). In some embodiments, the chemical name of the CELMOD can be 3-(5-Amino-2-methyl-4-oxo-4H-quinazolin-3-yl)piperidine-2,6-dione, or be 2,6-Piperidinedione,3-(5-amino-2-methyl-4-oxo-3(4H)-quinazolinyl)-. In some embodiments, the structural formula of the CELMOD can be

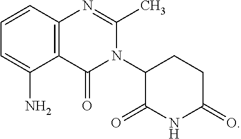 [SMILES: Cc1nc2cccc(N)c2c(=O)n1C1CCC(=O)NC1=O]

In some embodiments, the CELMOD can be administered orally at a dose of at least about 2 or 3 mg. In some embodiments, the CELMOD can be in a unit dose of at least about: 0.5-1 mg, 1-2 mg, 1-3 mg, 1-4 mg, 1-5 mg, 1-10 mg, 2-3 mg, 2-4 mg, 2-5 mg, 2-6 mg, 2-7 mg, 2-8 mg, 2-9 mg, 2-10 mg, 3-4 mg, 3-5 mg, 3-6 mg, 3-7 mg, 3-8 mg, 3-9 mg, 3-10 mg, 4-5 mg, 6-7 mg, 8-9 mg, 9-10 mg, 6-8 mg, 6-9 mg, or 6-10 mg.

### Dosage

In some cases, provided herein are combinations comprising a therapeutically effective dose of an anti-BCMA antigen binding protein comprising CDRH1 according to SEQ ID NO:1; CDRH2 according to SEQ ID NO:2; CDRH3 according to SEQ ID NO:3; CDRL1 according to SEQ ID NO:4; CDRL2 according to SEQ ID NO:5; and CDRL3 according to SEQ ID NO:6; and a CELMOD, for use in the treatment of cancer.

In some cases, provided herein are combinations comprising a therapeutically effective dose of an anti-BCMA antigen binding protein comprising heavy chain variable region (VH) according to SEQ ID NO:7; and a light chain variable region (VL) according to SEQ ID NO:8; and a CELMOD, for use in the treatment of cancer.

In some cases, provided herein are combinations comprising a therapeutically effective dose of an anti-BCMA antigen binding protein comprising heavy chain (H) according to SEQ ID NO:9 and a light chain (L) according to SEQ ID NO: 10; and a CELMOD, for use in the treating cancer.

In some embodiments, an anti-BCMA antigen binding protein disclosed herein comprises the heavy chain variable region of SEQ ID NO: 11, 15 or 19. In some embodiments, an anti-BCMA antigen binding protein disclosed herein comprises the light chain variable region of SEQ ID NO: 12, 16 or 20. In some embodiments, an anti-BCMA antigen binding protein disclosed herein comprises the heavy chain region of SEQ ID NO: 13, 17, 22, or 24. In some embodiments, an anti-BCMA antigen binding protein disclosed herein comprises the light chain region of SEQ ID NO: 14, 18, 23 or 25. In some embodiments, an anti-BCMA antigen binding protein disclosed herein comprises the heavy chain variable region of SEQ ID NO: 11 and the light chain variable region of SEQ ID NO: 12, the heavy chain variable region of SEQ ID NO: 15 and the light chain variable region of SEQ ID NO: 16, or the heavy chain variable region of SEQ ID NO: 19 and the light chain variable region of SEQ ID NO: 20. In some embodiments, an anti-BCMA antigen binding protein disclosed herein comprises the heavy chain region of SEQ ID NO: 13 and the light chain of SEQ ID NO: 14, the heavy chain region of SEQ ID NO: 17 and the light chain of SEQ ID NO: 18, the heavy chain region of SEQ ID NO: 22 and the light chain of SEQ ID NO: 23 or the heavy chain region of SEQ ID NO: 24 and the light chain of SEQ ID NO: 25. In some embodiments, an anti-BCMA antigen binding protein disclosed herein is an scFV-fc comprising SEQ ID NO: 21.

In some cases, a combination disclosed herein, when in a pharmaceutical preparation, can be present in unit dose form. In some embodiments, the dosage regimen will be determined by a medical profession and/or clinical factors. As is well known in the medical arts, dosages for any one patient depend upon many factors, including the patient's size, body surface area, age, the combination to be administered, sex, time and route of administration, general health, and other drugs being administered concurrently. Exemplary doses can vary according to the size and health of the individual being treated, as well as the condition being treated.

Suitable doses of the anti-BCMA antigen binding proteins described herein may be calculated for patients according to their weight, for example suitable doses may be in the range of about 0.1 mg/kg to about 20 mg/kg, for example about 1 mg/kg to about 20 mg/kg, for example about 10 mg/kg to about 20 mg/kg or for example about 1 mg/kg to about 15 mg/kg, for example about 10 mg/kg to about 15 mg/kg.

In some cases, the therapeutically effective dose of the anti-BCMA antigen binding protein can be in the range of about 0.03 mg/kg to about 4.6 mg/kg. In yet another embodiment, the therapeutically effective dose of the anti-BCMA antigen binding protein can be about or at least: 0.03 mg/kg, 0.06 mg/kg, 0.12 mg/kg, 0.24 mg/kg, 0.48 mg/kg, 0.96 mg/kg, 1.4 mg/kg, 1.92 mg/kg, 2.5 mg/kg, 3.4 mg/kg, or 4.6 mg/kg. In yet another embodiment, the therapeutically effective dose of the anti-BCMA antigen binding protein can be 1.4 mg/kg, 1.9 mg/kg, 2.5 mg/kg, or 3.4 mg/kg.

In some cases, the subject has received at least one previous cancer treatment. In some cases, the therapeutically effective dose of the combination can be administered to the subject at least about once every 1-60 days. In some cases, the therapeutically effective dose of the composition can be administered to the subject at least about once every 3, 4, 6, or 8 weeks (e.g., 21 days). In some cases, the therapeutically effective dose of the composition can be administered to the subject at least about once every 8 days.

### Route of Administration

In some cases, (i) an anti-BCMA antigen binding protein or an ADC having binding specificity for a BCMA polypeptide and (ii) one or more CELMODs can be administered to a mammal at the same time (e.g., in a single composition). In some cases, (i) an anti-BCMA antigen binding protein or an ADC having binding specificity for a BCMA polypeptide and (ii) one or more CELMODs can be administered to a subject separately. When administered separately, this may occur simultaneously or sequentially in any order (by the same or by different routes of administration). Such sequential administration may be close in time or remote in time. The dose of a therapeutic agents of the combination or pharmaceutical composition thereof and the further therapeutically active agent(s) and the relative timings of administration will be selected in order to achieve the desired combined therapeutic effect.

In some embodiments, the dosage can be administered a single time or multiple times, for example daily, weekly, biweekly, or monthly, hourly, or is administered upon recurrence, relapse or progression of a disease or condition being treated. In some embodiments, administration of a dose may be by slow continuous infusion over a period of from about 2 to about 24 hours, such as from about 2 to about 12 hours, or from about 2 to about 6 hours.

In some embodiments, a pharmaceutical composition disclosed herein can comprise the combination for parenteral, transdermal, intraluminal, intraarterial, intrathecal and/or intranasal administration or by direct injection into tissue. In some embodiments, the pharmaceutical composition can be administered to a patient via infusion or injection. In one embodiment, provided are pharmaceutical compositions comprising a BCMA binding protein and a CELMOD for intravenous administration. In some embodiments, provided are pharmaceutical compositions comprising a BCMA binding protein and a CELMOD for subcutaneous administration. In some embodiments, a pharmaceutical composition described herein can be administered to a subject transarterially, subcutaneously, intradermally, intratumorally, intranodally, intramedullary, intramuscularly, by intravenous (i.v.) injection, by intravenous (i.v.) infusion, or intraperitoneally. In some embodiments, the combination can be administered to a subject by intradermal or subcutaneous injection.

In one embodiment, one or more therapeutic agents of a combination can be administered intravenously. In another embodiment, one or more therapeutic agents of a combination can be administered intratumorally. In another embodiment, one or more therapeutic agents of a combination can be administered orally. In another embodiment, one or more therapeutic agents of a combination can be administered systemically, e.g., intravenously, and one or more other therapeutic agents of a combination of can be administered intratumorally. In another embodiment, all therapeutic agents of a combination disclosed herein can be administered systemically, e.g., intravenously. In an alternative embodiment, all therapeutic agents of the combination described herein can be administered intratumorally. In any of the embodiments, e.g., in this paragraph, the therapeutic agents disclosed herein can be administered as one or more pharmaceutical compositions.

### Pharmaceutical Composition

In some embodiments, a pharmaceutical composition can be prepared by per se known methods for the preparation of pharmaceutically acceptable compositions that are administered to subjects, such that an effective quantity of a BCMA binding protein+CELMOD is combined in a mixture with a pharmaceutically acceptable carrier. Suitable carriers are described, for example, in Remington's Pharmaceutical Sciences. On this basis, the compositions may include, albeit not exclusively, solutions of the substances in association with one or more pharmaceutically acceptable carriers or diluents, contained in buffered solutions with a suitable pH and iso-osmotic with the physiological fluids. In some embodiments, a pharmaceutical composition disclosed herein can be acidic. In some embodiments, a pharmaceutical composition disclosed herein can be basic. In some embodiments, a pharmaceutical composition can have a pH of about: 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, or about 14.

In some embodiments, suitable pharmaceutically acceptable carriers can include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, N-(1(2,3-dioleyloxy)propyl)N,N,N-trimethylammonium chloride (DOTMA), diolesylphosphotidyl-ethanolamine (DOPE), and liposomes. In some embodiments, such compositions contain a therapeutically effective amount of a BCMA binding protein and CELMOD disclosed herein, together with a suitable amount of carrier so as to provide the form for direct administration to a subject.

Pharmaceutical compositions may include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, preservatives, polyols, glycerin and vegetable oils, for example. Extemporaneous injection solutions and suspensions may be prepared from sterile powders, granules, tablets, or concentrated solutions or suspensions.

A pharmaceutical composition disclosed herein may be formulated into a variety of forms and administered by a number of different means. A pharmaceutical formulation can be administered orally, rectally, or parenterally, in formulations containing conventionally acceptable carriers, adjuvants, and vehicles as desired. The term “parenteral” as used herein includes subcutaneous, intravenous, intramuscular, or intrasternal injection and infusion techniques. Administration includes injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration. In some exemplary embodiments, a route of administration can be via an injection such as an intramuscular, intravenous, subcutaneous, or intraperitoneal injection.

Liquid formulations may include an oral formulation, an intravenous formulation, an intranasal formulation, an ocular formulation, an otic formulation, an aerosol, and the like. In certain embodiments, a combination of various formulations can be administered. In certain embodiments a composition can be formulated for an extended release profile.

Pharmaceutical compositions disclosed herein can be administered in combination with other therapeutics or treatments. In some embodiments, a treatment for a subject can be a surgery, radiation, chemotherapy, a nutrition regime, a physical activity, an immunotherapy, a pharmaceutical composition, a cell transplantation, a blood fusion, or any combination thereof. In some cases, the combination disclosed herein can be administered to a mammal having cancer together with one or more additional agents/therapies used to treat cancer. Examples of additional agents/therapies used to treat cancer include, without limitation, surgery, radiation therapies, chemotherapies, targeted therapies (e.g., monoclonal antibody therapies), hormonal therapies, angiogenesis inhibitors, immunosuppressants, checkpoint blockade therapies (e.g., anti-PD-1 antibody therapy, anti-PD-L1 antibody therapy, and/or anti-CTLA4 antibody therapy), bone marrow transplants.

In some embodiments, an additional cancer therapeutic may be carfilzomib, daratumumab, isatuximab, ixazomib, oprozomib, marizomib, or a pharmaceutically acceptable salt thereof. In some embodiments, an additional cancer therapeutic agent is a PD-1 inhibitor. In some cases, the PD-1 inhibitor is selected from the group consisting of PDR001, Nivolumab, Pembrolizumab, Pidilizumab, MEDI0680, REGN2810, TSR-042, PF-06801591, and AMP-224. In some cases, the PD-1 inhibitor is Jemperli. In some embodiments, an additional cancer therapeutic agent is a PD-L1 inhibitor. In some cases, the PD-L1 inhibitor is selected from the group consisting of FAZ053, Atezolizumab, Avelumab, Durvalumab, and BMS-93655. In some embodiments, an additional cancer therapeutic agent is a CTLA-4 inhibitor. In some cases, the CTLA-4 inhibitor is Ipilimumab or Tremelimumab. In some cases, an additional cancer therapeutic agent is a TIM⁻³ inhibitor. In some cases, the TIM⁻³ inhibitor is MGB453 or TSR-022. In some embodiments, an additional cancer therapeutic agent is a LAG-3 inhibitor. In some cases the LAG-3 inhibitor is selected from the group consisting of LAG525, BMS-986016, and TSR-033. In some embodiments, an additional cancer therapeutic agent is an mTOR inhibitor. In some cases the mTOR inhibitor is RAD001 or rapamycin.

In some embodiments, a combination/formulation disclosed herein can be stable. In some embodiments, a “stable” formulation is one in which the combination therein essentially retains its physical and/or chemical stability and/or biological activity upon storage. Stability can be measured at a selected temperature for a selected time period. In some embodiments, the formulation can be stable at ambient temperature or at 40° C. for at least 1 month and/or stable at 2-8° C. for at least 1 to 2 years. In some embodiments, the formulation can be stable following freezing (e.g., to −70° C.) and thawing. In some embodiments, a protein “retains its physical stability” in a formulation if it shows little to no change in aggregation, precipitation and/or denaturation as observed by visual examination of color and/or clarity, or as measured by UV light scattering (measures visible aggregates) or size exclusion chromatography (SEC). SEC measures soluble aggregates that are not necessarily a precursor for visible aggregates. In some embodiments, a protein “retains its chemical stability” in a formulation if the chemical stability at a given time is such that the protein is considered to retain its biological activity. Chemically degraded species may be biologically active and chemically unstable. Chemical stability can be assessed by detecting and quantifying chemically altered forms of the protein. Chemical alteration may involve size modification (e.g., clipping) which can be evaluated using SEC, SDS-PAGE and/or matrix-assisted laser desorption ionization/time-of-flight mass spectrometry (MALDI/TOF MS), for example. Other types of chemical alteration include charge alteration (e.g., occurring as a result of deamidation) which can be evaluated by ion-exchange chromatography, for example.

In some embodiments, a BCMA binding protein “retains its biological activity” in a pharmaceutical formulation, if the biological activity of the BCMA binding protein at a given time is within about 10% (within the errors of the assay) of the biological activity exhibited at the time the pharmaceutical formulation was prepared as determined in an antigen binding assay, for example. In a some embodiments, a CELMOD “retains its biological activity” in a pharmaceutical formulation, if the biological activity of the CELMOD at a given time is within about 10% (within the errors of the assay) of the biological activity exhibited at the time the pharmaceutical formulation was prepared as determined in an antigen binding assay, for example.

In some embodiments, a buffer disclosed herein refers to a buffered solution that resists changes in pH by the action of its acid-base conjugate components. In some embodiments, a buffer can be phosphate, citrate and other organic acids. In some embodiments, a buffer can be selected from the group consisting of sodium acetate, sodium carbonate, citrate, glycylglycine, histidine, glycine, lysine, arginine, sodium dihydrogen phosphate, disodium hydrogen phosphate, sodium phosphate, sodium citrate, sodium borate, tris(hydroxymethyl)-aminomethan, bicine, tricine, malic acid, succinate, maleic acid, fumaric acid, tartaric acid, aspartic acid or mixtures thereof. A composition disclosed herein can comprise antioxidants including ascorbic acid and/or methionine. In some embodiments, a composition disclosed herein can comprise a preservative. In some embodiments, a preservative can be a compound which can be included in a formulation to essentially reduce microbial including bacterial action therein, thus facilitating the production of a multi-use formulation, for example. Examples of potential preservatives include octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, a combination disclosed herein may further comprise a chemotherapeutic agent, cytotoxic agent, cytokine, growth inhibitory agent, anti-hormonal agent, and/or cardioprotectant. Such molecules can be present in combination in amounts that are effective for the purpose intended.

In some embodiments, a combination disclosed herein can be prepared in a sustained-release preparation. Suitable examples of sustained-release preparations include semipermeable matrices of solid hydrophobic polymers containing the combination or portions thereof, which matrices can be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

In some embodiments, disclosed herein are pharmaceutical compositions comprising a BCMA binding protein and a CELMOD which can be present in a concentration from 1 mg/ml to 500 mg/ml, and wherein the pharmaceutical composition has a pH from 2.0 to 10.0. The pharmaceutical composition may further comprise a buffer system, preservative(s), tonicity agent(s), chelating agent(s), stabilizers and surfactants. In some embodiments, the pharmaceutical composition can be an aqueous formulation, for example, formulation comprising water. Such formulation can be a solution or a suspension. In a further embodiment, the pharmaceutical formulation can be an aqueous solution. In some embodiments, an aqueous formulation can be a formulation comprising at least 50% w/w water. In some embodiments an aqueous solution is defined as a solution comprising at least 50% w/w water. In some embodiments, the pharmaceutical composition can be a stable liquid aqueous pharmaceutical formulation comprising a combination described herein.

The pharmaceutical compositions may also comprise additional stabilizing agents, that may further enhance stability of a therapeutically active combination. Stabilizing agents of can include, but are not limited to, methionine and EDTA, which protect polypeptides against methionine oxidation, and a nonionic surfactant, which protects polypeptides against aggregation associated with freeze-thawing or mechanical shearing. In some embodiments, the pharmaceutical composition may further comprise a surfactant. The surfactant may be selected from a detergent, ethoxylated castor oil, polyglycolyzed glycerides, acetylated monoglycerides, sorbitan fatty acid esters, polyoxypropylene-polyoxyethylene block polymers (eg. poloxamers such as PLURONIC F68, poloxamer 188 and 407, Triton X-100), polyoxyethylene sorbitan fatty acid esters, polyoxyethylene and polyethylene derivatives such as alkylated and alkoxylated derivatives (tweens, e.g., Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and I-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g., lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives- (e.g., sodium tauro-dihydrofusidate etc.), long-chain fatty acids and salts thereof C6-C12 (eg. oleic acid and caprylic acid), acylcarnitines and derivatives, N^{a}-acylated derivatives of lysine, arginine or histidine, or side-chain acylated derivatives of lysine or arginine, N^{a}-acylated derivatives of dipeptides comprising any combination of lysine, arginine or histidine and a neutral or acidic amino acid, N^{a}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-I-propanesulfonate, anionic (alkyl-aryl-sulphonates) monovalent surfactants, zwitterionic surfactants (e.g., N-alkyl-N,N-dimethylammonio-I-propanesulfonates, 3-cholamido-I-propyldimethylammonio-I-propanesulfonate, cationic surfactants (quaternary ammonium bases) (e.g., cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof.

### Kits

A kit-of-parts comprising a pharmaceutical composition together with instructions for use is further provided. For convenience, the kit-of-parts may comprise reagents in predetermined amounts with instructions for use.

In some embodiments, disclosed herein are kits comprising a BCMA binding protein and a CELMOD disclosed herein. A kit may include a plurality of syringes, ampules, foil packets, or blister packs, each containing a single unit dose of a kit component described herein. Containers of a kit may be airtight, waterproof (e.g., impermeable to changes in moisture or evaporation), and/or light-tight. A kit may include a device suitable for administration of the component, e.g., a syringe, inhalant, pipette, forceps, measured spoon, dropper (e.g., eye dropper), swab (e.g., a cotton swab or wooden swab), or any such delivery device. In some embodiments, the device may be a medical implant device, e.g., packaged for surgical insertion. A kit disclosed herein may comprise one or more reagents or instruments which enable the method to be carried out.

In addition to the above components, instructions for use may be provided in a kit. These instructions may be present in the kit in a variety of forms, such as printed information on a suitable medium or substrate (e.g., a piece or pieces of paper on which the information is printed), in the packaging of the kit, in a package insert, etc. In some embodiments, instructions for use can be provided on a computer readable medium (e.g., jump/thumb drive, CD, etc.), on which the information has been recorded or at a website address which may be used via the internet to access the information at a website.

### Devices

Another aspect of the present disclosure provides a pre-filled syringe or autoinjector device, comprising a BCMA antigen binding protein, a CELMOD or a combination described herein. In some embodiments, a combination stored in a container, pre-filled syringe, injector or autoinjector device can contain a BCMA antigen binding protein and a CELMOD disclosed herein.

### EXAMPLES

### Example 1: Treating Cancer

A subject will be identified as having cancer. The subject will be administered a combination comprising (a) an anti-BCMA antigen binding protein or an anti-BCMA ADC and (b) a CELMOD.

### Example 2: Treating Cancer

A subject will be identified as having cancer. The subject will be administered (a) an anti-BCMA antigen binding protein or an anti-BCMA ADC and (b) a CELMOD in separate compositions that will be co-administered. For example, the subject having cancer will be co-administered a first composition including one or more ADCs having binding specificity for a BCMA polypeptide and a second composition including one or more CELMODs.

### Example 3: Treating Cancer

A subject will be identified as having cancer. The subject will be administered (a) an anti-BCMA antigen binding protein or an anti-BCMA ADC and (b) a CELMOD in separate compositions that will be separately administered. For example, the subject having cancer will be separately administered a first composition including one or more ADCs having binding specificity for a BCMA polypeptide and a second composition including one or more CELMODs.

### Example 4: Treating Cancer

A subject will be identified as having cancer. The subject will be administered a combination comprising 1) belantamab mafodotin and 2) mezigdomide (CC-92480), iberdomide (CC-220), avadomide (CC-122), CC-90009, CC-99282, or a pharmaceutically acceptable salt thereof.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Embodiments

1. A combination comprising:
   a. an anti-BCMA antigen binding protein; and
   b. a cereblon E3 ligase modulator (CELMOD).
2. The combination of embodiment 1, wherein the anti-BCMA antigen binding protein comprises an antibody.
3. The combination of embodiment 2, wherein the antibody is a monoclonal antibody.
4. The combination of embodiment 3, wherein the monoclonal antibody is an IgG1.
5. The combination of any one of embodiments 2-4, wherein the antibody is afucosylated.
6. The combination of any one of embodiments 1-5, wherein the anti-BCMA antigen binding protein is human, humanized, or chimeric.
7. The combination of any one of embodiments 1-6, wherein the anti-BCMA antigen binding protein comprises a CDRH1 comprising the amino acid sequence set out in SEQ ID NO:1; a CDRH2 comprising the amino acid sequence set out in SEQ ID NO:2; a CDRH3 comprising the amino acid sequence set out in SEQ ID NO:3; a CDRL1 comprising the amino acid sequence set out in SEQ ID NO:4; a CDRL2 comprising the amino acid sequence set out in SEQ ID NO:5; and a CDRL3 comprising the amino acid sequence set out in SEQ ID NO:6.
8. The combination of any one of embodiments 1-7, wherein the anti-BCMA antigen binding protein comprises a heavy chain variable region (VH) comprising the amino acid sequence set out in SEQ ID NO:7; and a light chain variable region (VL) comprising the amino acid sequence set out in SEQ ID NO:8.
9. The combination of any one of embodiments 1-8, wherein the anti-BCMA antigen binding protein comprises a heavy chain (H) comprising the amino acid sequence set out in SEQ ID NO: 9 and a light chain (L) comprising the amino acid sequence set out in SEQ ID NO: 10.
10. The combination of any one of embodiments 1-9, wherein the anti-BCMA antigen binding protein is an immunoconjugate.
11. The combination of any one of embodiments 1-10, wherein the anti-BCMA antigen binding protein is an immunoconjugate comprising an antibody conjugated to a cytotoxin.
12. The combination of embodiment 11, wherein the cytotoxin is MMAE or MMAF.
13. The combination of embodiment 12, wherein the cytotoxin is MMAF.
14. The combination of any one of embodiments 1-13, wherein the anti-BCMA antigen binding protein is belantamab mafodotin.
15. The combination of embodiment 14, wherein the combination comprises at least about: 0.95 mg/kg, 1.9 mg/kg, 1.4 mg/kg, 2.5 mg/kg or 3.4 mg/kg belantamab mafodotin.
16. The combination of any one of embodiments 1-15, wherein the cereblon E3 ligase modulator has a molecular weight less than 1000 Dalton.
17. The combination of any one of embodiments 1-16, wherein the cereblon E3 ligase modulator degrades an Ikaros protein, an Aiolos protein, or a combination thereof.
18. The combination of any one of embodiments 1-17, wherein the cereblon E3 ligase modulator has an IC₅₀ value less than 1 pM to cereblon.
19. The combination of any one of embodiments 1-18, wherein the cereblon E3 ligase modulator comprises mezigdomide (CC-92480), iberdomide (CC-220), avadomide (CC-122), CC-90009, CC-99282, a pharmaceutically acceptable salt of any of the foregoing, or any combination thereof.
20. The combination of any one of embodiments 1-18, wherein the cereblon E3 ligase modulator is mezigdomide or a pharmaceutically acceptable salt thereof.
21. The combination of embodiment 20, wherein the combination comprises at least about: 0.1 mg, 0.2 mg, 0.3 mg, 0.4 mg, 0.5 mg, 0.6 mg, 0.7 mg, 0.8 mg, 0.9 mg, or 1 mg of mezigdomide or the pharmaceutically acceptable salt thereof.
22. The combination of any one of embodiments 1-18, wherein the cereblon E3 ligase modulator is iberdomide or a pharmaceutically acceptable salt thereof.
23. The combination of embodiment 22, wherein the combination comprises at least about: 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, or 1.3 mg of iberdomide or the pharmaceutically acceptable salt thereof.
24. The combination of any one of embodiments 1-18, wherein the cereblon E3 ligase modulator degrades GSPT1.
25. The combination of embodiment 24, wherein the cereblon E3 ligase modulator is CC-90009 or a pharmaceutically acceptable salt thereof.
26. The combination of any one of embodiments 1-18, wherein the cereblon E3 ligase modulator is avadomide or a pharmaceutically acceptable salt thereof.
27. The combination of any one of embodiments 1-18, wherein the cereblon E3 ligase modulator is CC-99282 or a pharmaceutically acceptable salt thereof.
28. The combination of any one of embodiments 1-27, wherein the combination further comprises a pharmaceutically acceptable carrier.
29. The combination of any one of embodiments 1-28, wherein the combination further comprises an adjuvant.
30. The combination of any one of embodiments 1-29, wherein the combination is more synergistic in anti-cancer activity than a combination of the anti-BCMA antigen binding protein and an immunomodulatory drug (IMiD).
31. The combination of embodiment 30, wherein the MiD comprises bortezomib, pomalidomide, lenalidomide, dexamethasone, thalidomide, or a pharmaceutically acceptable salt thereof.
32. A method of treating a cancer in a subject in need thereof, comprising administering to the subject a therapeutically effective dose of the combination of any one of embodiments 1-31.
33. The method of embodiment 32, wherein the cancer is selected from the group consisting of multiple myeloma, chronic lymphocytic leukemia, Waldenstrom macroglobulinemia, and non-Hodgkin's lymphoma.
34. The method of embodiment 32 or 33, wherein the cancer is multiple myeloma.
35. The method of embodiment 34, wherein the cancer is relapsed and/or refractory multiple myeloma.
36. The method of any one of embodiments 32-35, wherein the subject has received at least one previous cancer treatment.
37. The method of any one of embodiments 32-36, wherein the therapeutically effective dose is administered to the subject at least about once every 3, 4, 6, or 8 weeks.
38. The method of any one of embodiments 32-37, wherein the administering the therapeutically effective dose of the combination reduces ocular toxicity as compared to administering a therapeutically effective amount of the anti-BCMA antigen binding protein alone.
39. The method of embodiment 38, wherein the anti-BCMA antigen binding protein is belantamab mafodotin.
40. The method of embodiment 38 or 39, wherein the ocular toxicity is at least one of: changes in corneal epithelium, dry eyes, irritation, redness, blurred vision, dry eyes, photophobia, or changes in visual acuity.
41. The method of any one of embodiments 38-40, wherein the ocular toxicity is measured by at least one of the following methods: best corrected visual acuity, documentation of manifest refraction and the method used to obtain best corrected visual acuity, current glasses prescription (if applicable), intraocular pressure measurement, anterior segment (slit lamp) examination including fluorescein staining of the cornea and lens examination, dilated funduscopic examination, or an ocular surface disease index (OSDI).
42. The method of any one of embodiments 32-41, wherein the anti-BCMA antigen binding protein is administered to the subject in a dose of at least about: 0.5 mg/kg, 0.95 mg/kg, 1.25 mg/kg, 1.7 mg/kg, 2.5 mg/kg, or 3.4 mg/kg.
43. A combination of any one of embodiments 1-31, for use in the manufacture of a medicament for treatment of cancer.
44. A combination of any one of embodiments 1-31, for use in treatment of a cancer.
45. A kit for use in treatment of a cancer comprising:
   a. the combination of any one of embodiments 1-31; and
   b. instructions for use in the treatment of cancer.
46. A pre-filled syringe or autoinjector device, comprising the combination of any one of embodiments 1-31.

1. A combination comprising:
a. an anti-BCMA antigen binding protein; and
b. a cereblon E3 ligase modulator (CELMOD).

2. The combination of claim 1, wherein the anti-BCMA antigen binding protein comprises an antibody.

3.-6. (canceled)

7. The combination of claim 1, wherein the anti-BCMA antigen binding protein comprises a CDRH1 comprising the amino acid sequence set out in SEQ ID NO:1; a CDRH2 comprising the amino acid sequence set out in SEQ ID NO:2; a CDRH3 comprising the amino acid sequence set out in SEQ ID NO:3; a CDRL1 comprising the amino acid sequence set out in SEQ ID NO:4; a CDRL2 comprising the amino acid sequence set out in SEQ ID NO:5; and a CDRL3 comprising the amino acid sequence set out in SEQ ID NO: 6.

8. The combination of claim 1, wherein the anti-BCMA antigen binding protein comprises a heavy chain variable region (VH) comprising the amino acid sequence set out in SEQ ID NO:7; and a light chain variable region (VL) comprising the amino acid sequence set out in SEQ ID NO:8.

9. The combination of claim 1, wherein the anti-BCMA antigen binding protein comprises a heavy chain (H) comprising the amino acid sequence set out in SEQ ID NO: 9 and a light chain (L) comprising the amino acid sequence set out in SEQ ID NO: 10.

10. (canceled)

11. The combination of claim 1, wherein the anti-BCMA antigen binding protein is an immunoconjugate comprising an antibody conjugated to a cytotoxin.

12. The combination of claim 11, wherein the cytotoxin is MMAE or MMAF.

13. (canceled)

14. The combination of claim 1, wherein the anti-BCMA antigen binding protein is belantamab mafodotin.

15. The combination of claim 14, wherein the combination comprises at least about: 0.95 mg/kg, 1.4 mg/kg, 1.9 mg/kg, 2.5 mg/kg, or 3.4 mg/kg belantamab mafodotin.

16. (canceled)

17. The combination of claim 1, wherein the cereblon E3 ligase modulator degrades an Ikaros protein, an Aiolos protein, GSPT1, or a combination thereof.

18. (canceled)

19. The combination of claim 1, wherein the cereblon E3 ligase modulator comprises mezigdomide (CC-92480), iberdomide (CC-220), avadomide (CC-122), CC-90009, CC-99282, a pharmaceutically acceptable salt of any of the foregoing, or any combination thereof.

20. The combination of claim 1, wherein the cereblon E3 ligase modulator is mezigdomide or a pharmaceutically acceptable salt thereof.

21. (canceled)

22. The combination of claim 1, wherein the cereblon E3 ligase modulator is iberdomide or a pharmaceutically acceptable salt thereof.

23.-31. (canceled)

32. A method of treating a cancer in a subject in need thereof, comprising administering to the subject a therapeutically effective dose of the combination of claim 1.

33. The method of claim 32, wherein the cancer is selected from the group consisting of multiple myeloma, chronic lymphocytic leukemia, Waldenstrom macroglobulinemia, and non-Hodgkin's lymphoma.

34. The method of claim 32, wherein the cancer is multiple myeloma.

35. The method of claim 34, wherein the cancer is relapsed and/or refractory multiple myeloma.

36. The method of claim 32, wherein the subject has received at least one previous cancer treatment.

37. (canceled)

38. The method of claim 32, wherein the administering the therapeutically effective dose of the combination reduces ocular toxicity as compared to administering a therapeutically effective amount of the anti-BCMA antigen binding protein alone.

39. The method of claim 38, wherein the anti-BCMA antigen binding protein is belantamab mafodotin.

40.-46. (canceled)


SEQUENCE LISTING (25 sequences)

SEQ ID NO: 1 - protein, 5 aa, synthetic construct
  source 1..5: note = CDR sequences for an anti-BCMA antigen binding protein VH CDR1
NYWMH

SEQ ID NO: 2 - protein, 17 aa, synthetic construct
  source 1..17: note = CDR sequences for an anti-BCMA antigen binding protein VH CDR2
ATYRGHSDTYYNQKFKG

SEQ ID NO: 3 - protein, 12 aa, synthetic construct
  source 1..12: note = CDR sequences for an anti-BCMA antigen binding protein VH CDR3
GAIYDGYDVLDN

SEQ ID NO: 4 - protein, 11 aa, synthetic construct
  source 1..11: note = CDR sequences for an anti-BCMA antigen binding protein VL CDR1
SASQDISNYLN

SEQ ID NO: 5 - protein, 7 aa, synthetic construct
  source 1..7: note = CDR sequences for an anti-BCMA antigen binding protein VL CDR2
YTSNLHS

SEQ ID NO: 6 - protein, 9 aa, synthetic construct
  source 1..9: note = CDR sequences for an anti-BCMA antigen binding protein VL CDR3
QQYRKLPWT

SEQ ID NO: 7 - protein, 121 aa, synthetic construct
  source 1..121: note = heavy chain variable region
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 8 - protein, 108 aa, synthetic construct
  source 1..108: note = light chain variable region
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 9 - protein, 451 aa, synthetic construct
  source 1..451: note = heavy chain region
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
  source 1..214: note = light chain region
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 119 aa, synthetic construct
  source 1..119: note = BQ76 heavy chain variable region
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVAPYFAPFDYWGQGTLVTVSS

SEQ ID NO: 12 - protein, 109 aa, synthetic construct
  source 1..109: note = BQ76 light chain variable region
EIVLTQSPGTLSLSPGERATLSCRASQSVSSSYLAWYQQKPGQAPRLLIYGASSRATGIPDRFSGSGSGTDFTLTISRLEPEDFAVYYCQQYGNPPLYTFGQGTKVEIK

SEQ ID NO: 13 - protein, 449 aa, synthetic construct
  source 1..449: note = BQ76 heavy chain region
  MOD_RES 449: note = X is K or absent
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVAPYFAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGX

SEQ ID NO: 14 - protein, 216 aa, synthetic construct
  source 1..216: note = BQ76 light chain regionv
EIVLTQSPGTLSLSPGERATLSCRASQSVSSSYLAWYQQKPGQAPRLLIYGASSRATGIPDRFSGSGSGTDFTLTISRLEPEDFAVYYCQQYGNPPLYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 117 aa, synthetic construct
  source 1..117: note = BU76 heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 16 - protein, 111 aa, synthetic construct
  source 1..111: note = BU76 light chain variable region
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 17 - protein, 447 aa, synthetic construct
  source 1..447: note = BU76 heavy chain region
  MOD_RES 447: note = X is K or absent
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGX

SEQ ID NO: 18 - protein, 218 aa, synthetic construct
  source 1..218: note = BU76 light chain region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 116 aa, synthetic construct
  source 1..116: note = EE11 heavy chain variable region
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSS

SEQ ID NO: 20 - protein, 109 aa, synthetic construct
  source 1..109: note = EE11 light chain variable region
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIK

SEQ ID NO: 21 - protein, 477 aa, synthetic construct
  MOD_RES 477: note = X is K or absent
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIKGGGGSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 22 - protein, 449 aa, synthetic construct
  source 1..449: note = EM90 heavy chain
  MOD_RES 449: note = X is K or absent
EVQLVESGGGLVKPGGSLRLSCAASGFTFSNSGMIWVRQAPGKGLEWVGHIRSKTDGGTTDYAAPVKGRFTISRDDSKNTLYLQMNSLKTEDTAVYYCTTGGSGSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGX

SEQ ID NO: 23 - protein, 215 aa, synthetic construct
  source 1..215: note = EM90 light chain
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 24 - protein, 441 aa, synthetic construct
  source 1..441: note = FP31 heavy chain region
EVQLLESGGGLVQPGGSLRLSCAASGFTFSSYPMSWVRQAPGKGLEWVSAIGGSGGSLPYADSVKGRFTISRDNSKNTLYLQMNSLRAEDTAVYYCARYWPMDIWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCEVECPECPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCEVKGFYPSDISVEWESNGQPENNYKTTPPMLDSDGSFFLYSKLTVDKSRWQQGNVFSCSVMHEALHNHYTQKSLSLSPGK

SEQ ID NO: 25 - protein, 215 aa, synthetic construct
  source 1..215: note = FP31 light chain region
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC